(19) United States
(12) Patent Application Publication
(10) Pub. No.: US 2014/0066726 A1
(43) Pub. Date: Mar. 6, 2014

(54) Biological Sample Collection Device and System

(71) Applicant: Benedict Costello, Piedmont, CA (US)

(72) Inventor: Benedict Costello, Piedmont, CA (US)

(73) Assignee: PROTEUS DIGITAL HEALTH, INC., Redwood City, CA (US)

(21) Appl. No.: 14/004,395

(22) PCT Filed: Mar. 8, 2012
(86) PCT No.: PCT/US2012/028342

§ 371 Date: Nov. 18, 2013

Related U.S. Application Data
(60) Provisional application No. 61/451,934, filed on Mar. 11, 2011.

(51) Int. Cl. A61B 5/15; A61B 10/00; A61B 5/053; A61B 5/07; A61B 5/00; A61B 5/157

(52) U.S. Cl. CPC A61B 5/150091; A61B 5/150358; A61B 5/6832; A61B 5/150984; A61B 5/157; A61B 5/053; A61B 5/073; A61B 10/0064; A61B 5/686
USPC 600/302; 600/573; 600/367; 600/393

(57) ABSTRACT

A biological sample collection device includes a top cover plate, a bottom portion attached to the top cover plate, the bottom portion comprising a remotely-analyzable biological sample collection portion to collect a biological sample from a body of a subject. The biological sample is to be analyzed at a remote processing facility at a later time. A fastening portion is provided on the bottom portion to affix the remotely-analyzable biological sample collection device to the body of the subject. A real time clock is coupled to the top cover plate and a memory is coupled to the top cover plate and electrically coupled to the real time clock. The remotely-analyzable biological sample collection device may further include a processing unit, at least two electrodes coupled to the processing unit, and a transbody conductive communication module. Additionally, a remotely-analyzable biological sample collection device communication system includes a wireless communication device configured to communicate with a communication device external to the patient.

### CROSS REFERENCE TO RELATED APPLICATIONS

This application claims the benefit of U.S. Provisional Patent Application No. 61/451,934 entitled “Biological Sample Collection Device and System” and filed on Mar. 11, 2011, which is herein entirely incorporated by reference.

### INTRODUCTION

The present disclosure is related generally to a biological sample collection device for remote management of disease. In particular, the present disclosure is related to a wearable biological sample collection device for periodic sampling of analytes at a processing facility.

The remote management of disease, particularly chronic disease, requires information about the state of health of a subject. Some indications of the health of the subject can be derived from existing wearable diagnostic and monitoring devices. In particular, Holter recorders can inform a caregiver of the heart rate, sleep apnea monitors can record and communicate unusual breathing patterns, and these metrics plus the activity of the subject can be monitored by existing devices by Proteus Biomedical, Inc. of Redwood City, Calif. Some wearable diagnostic and monitoring devices are known as body-associated receivers or simply patches. Although the above described metrics are undeniably useful, and sometimes lifesaving, disease management also requires knowledge of chemical indicators of health. Examples of chemical health indices include the blood glucose of diabetics, creatinine levels in dialysis patients, or serum drug levels in many conditions. While there is ongoing extensive research on wearable chemical or biochemical sensors, few of these devices have made it out of the laboratory. Shortcomings of existing chemical/biochemical sensors include insufficient accuracy, inadequate specificity, poor repeatability, short operating life, complexity, and excessive cost.

### SUMMARY

In one aspect, a biological sample collection device includes a top cover plate, a bottom portion attached to the top cover plate, and a fastening portion provided on the bottom portion. The bottom portion includes a remotely-analyzable biological sample collection portion to collect a biological sample from a body of a subject. The biological sample is to be analyzed at a remote processing facility at a later time. The fastening portion affixes the remotely-analyzable biological sample collection device to the body of the subject. A real-time clock is coupled to the top cover plate and a memory also coupled to the top cover plate is electrically coupled to the real time clock.

In another aspect, a remotely-analyzable biological sample collection system further includes a processing unit, at least two electrodes, and a transbody conductive communication module. The at least two electrodes are coupled to the processing unit and are configured to contact the skin of a subject. The transbody conductive communication module is coupled to the processing unit and the at least two electrodes. The transbody conductive communication module is operative to detect and gather physiological information from the subject in the form of an electric current flow through the at least two electrodes at a first frequency. The current flow at the first frequency is associated with a device associated with the subject.

In yet another aspect, a remotely-analyzable biological sample collection system further includes a physiological sensing module coupled to the processing unit and a wireless communication module. The physiological sensing module is operative to sense physiological information from the subject in the form of electric current flow through the at least two electrodes at a second frequency. The current flow at the second frequency is associated with the physiology of the subject. The wireless communication module is coupled to the processing unit and is operative to communicate information from the remotely-analyzable biological collection device to a communication device external to the subject.

### FIGURES

FIG. 1 illustrates one aspect of a biological sample collection device positioned on a living subject.

FIG. 2 illustrates one aspect of a processing facility to receive a biological sample collection device for processing.

FIG. 3 illustrates one aspect of a biological sample collection device that comprises an adsorbent material.

FIG. 3A is a cross-sectional view of the biological sample collection device shown in FIG. 3 taken along line 3A-3A.

FIG. 4 illustrates one aspect of a biological sample collection device that comprises an absorbent material.

FIG. 4A is a cross-sectional view of the biological sample collection device shown in FIG. 4 taken along line 4A-4A.

FIG. 5 illustrates one aspect of a biological sample collection device that comprises an adhesive material.

FIG. 5A is a cross-sectional view of the biological sample collection device shown in FIG. 5 taken along line 5A-5A.

FIG. 6 illustrates one aspect of a biological sample collection device that comprises at least one micro-needle.

FIG. 6A is a cross-sectional view of the biological sample collection device shown in FIG. 6 taken along line 6A-6A.

FIG. 7 illustrates one aspect of a biological sample collection device that comprises electrodes and electronic components to extract body fluids from the subject via reverse electrophoresis.

FIG. 7A is a cross-sectional view of the biological sample collection device shown in FIG. 7 taken along line 7A-7A.

FIG. 8 illustrates one aspect of a biological sample collection device for detecting the presence or absence of a substance using a color indicator.

FIG. 8A is a cross-sectional view of the biological sample collection device shown in FIG. 8 taken along line 8A-8A.

FIG. 9 illustrates one aspect of a biological sample collection device system comprising a receiver portion and a biological sample collection portion.

FIG. 10 is a block functional diagram of one aspect of an integrated circuit component of a receiver component of the biological sample collection system shown in FIG. 9

FIG. 11 illustrates one aspect of an external biological sample collection system comprising a receiver portion and a biological sample collection portion.

FIG. 12 is an exploded view of the biological sample collection system shown in FIG. 11.

FIG. 13 is an exploded view of the adhesive patch portion of the biological sample collection system shown in FIG. 12.

FIGS. 14A to 14E illustrate various views of an alternative external biological sample collection system configuration which includes two electrodes in a flexible structure having an adhesive bandage configuration.

FIG. 15 illustrates one aspect of a communication system for a biological sample collection system.

FIG. 16 illustrates one aspect of a biological sample collection device comprising a framework for generating an operational voltage, a real time clock, and a memory.

### DESCRIPTION

The present specification describes multiple aspects of a biological sample collection device, biological sample collection system, and a communication system therefore, for remote management of disease. In one aspect, the biological sample collection device can be realized in the form of a patch that may be positioned on a subject. In various aspects, the biological sample collection device can be used for periodic sampling of biological or biochemical/chemical substance constituents, e.g., analytes, secreted by the subject and collected by the biological sample collection device over a predetermined period. The analytes can be determined using an analytical procedure at a processing facility. In various aspects, the biological sample collection device may be wearable, implantable, or semi-implantable on the or in the subject. In one aspect, the biological sample collection device may be combined in a system with receivers. In other aspects, the biological sample collection device may be combined with sensing and recording elements. In other aspects, the biological sample collection device may be combined in a communication system. Examples of communication systems include receivers to detect information from the subject encoded in current flows through a conducting solution and systems capable of communicating with one or more of the communication devices.

Various aspects of the disclosed biological sample collection device and systems may have particular utility in places where the cost of healthcare is a real challenge such as underdeveloped countries where continuous cell phone coverage and even going to a hospital to have a lab test may be impractical. The disclosed biological sample collection device and systems, however, provide a method for collecting physiological information and analyzing the information remotely so that a caregiver does not need to be located near the subject or even in the same continent as the subject.

FIG. 1 illustrates one aspect of a biological sample collection device 100 positioned on a living subject 102. In various aspects, the biological sample collection device may be wearable, implantable, or semi-implantable on the or in the subject 102. As shown in FIG. 1, the biological sample collection device 100 is externally affixed to the bare skin of the subject 102. The biological sample collection device 100 may be realized in passive or active configurations.

In a passive configuration, the biological sample collection device 100 includes passive elements that merely collect physiological information (e.g., physiological parameters or biomarkers) over a predetermined period for later analysis at a processing facility. The passive configuration does not employ sensors, receivers, electronic recording devices, memory, communication capabilities, or batteries. In one aspect, the biological sample collection device 100 may comprise one or more passive physiological parameter collection abilities. By physiological parameter collection ability is meant a capability of collecting a physiological parameter or biomarker including but not limited to: chemical composition of fluid, e.g., analyte in blood, perspiration (sweat), extra-cellular fluid, excreted oils, skin cells, hair follicles, viruses, bacteria, antibodies, DNA, molecules of various sizes associated with the subject 102. In various aspects, the biological sample collection device 100 may be configured to collect small molecules or elements like potassium, sodium, alcohol, nicotine, and other drugs of abuse to assist addicts; larger molecules like glucose and proteins like antibodies and DNA; and particles like viruses and bacteria. The term “biomarker” refers to an anatomic, physiologic, biochemical, or molecular parameter associated with the presence and severity of specific disease states.

In an active configuration, the biological sample collection device 100 comprises one or more active components to dynamically detect and gather physiological parameters in addition to passively collecting physiological parameters or biomarkers. In one aspect, active components include but are not limited to: sensors, receivers, electronic recording devices, memory, communication components. In an active configuration, the biological sample collection device 100 may include an on-board battery to supply electrical power to the active components. The biological sample collection device 100 may comprise a real time clock coupled to the cover plate to time-stamp (e.g., record in memory the date and/or time) when the data collection is actually performed. Active physiological parameter or biomarker sensing abilities include, without limitation, sensing: cardio-data, including heart rate, electrocardiogram (ECG), and the like; respiration rate, temperature; pressure; chemical composition of fluid, e.g., analyte in blood, fluid state, blood flow rate, accelerometer motion data.

The biological sample collection device 100 may comprise any number of distinct physiological parameter or biomarker collecting and/or sensing capabilities. The number of distinct parameters or biomarker collecting and/or sensing capabilities may vary e.g., one or more, two or more, three or more, four or more, five or more, ten or more, and so on.

The biological sample collection device 100 provides specific information about the state of health of the subject 102. Although some of this information may be derived from sensors embedded in the biological sample collection device 100 (e.g., active mode), there is a limitation in the sensor technology such that it is not practical to implement small ambulatory, inexpensive, biochemical/chemical sensors capable of being embedded in the biological sample collection device 100. As an example, there is no existing practical sensor for measuring viral load for a subject that is HIV positive and needs to control viral loads through entry retroviral medicines. Accordingly, instead of trying to embed sensors into a wearable device, the present disclosure provides a biological sample collection device 100 for collecting biological samples over a predetermined period, which then can be analyzed in a conventional way at a processing facility. The subject 102 may obtain the biological sample collection device 100 with a prescription, wear the biological sample collection device 100 for a prescribed period (e.g., minutes, hours, days, or weeks), and send it to a processing laboratory facility where the materials that were collected (e.g., biochemical/chemical samples) by the biological sample collection device 100 are removed and a suite of laboratory tests are conducted on the samples to measure any number of physiologic parameters and/or biomarkers or simply ascertain the presence or absence of a particular physiological parameter or biomarker. For instance, if the biological sample collection device 100 collected extra-cellular fluid, the laboratory could analyze the extra-cellular fluid for blood chemistry, glucose for the diabetics, creatinine for people on dialysis or recipients of organ transplant, and so on.

In one aspect, the subject 102 merely affixes the biological sample collection device 100 to the skin to collect raw physiological parameters or biomarkers (e.g., biological samples such as biochemicals/chemicals) in the field over several minutes, hours, days, weeks, or other suitable period. Since not all analytes require moment-by-moment measurement to optimize therapy, for those analytes for which periodic sampling is adequate, the limitations of biosensors can be overcome by configuring the biological sample collection device 100 not as an analysis tool but as a sample collection tool—a biological sample collection device. Once the relevant information is extracted from the biological sample collection device 100, the sample collection device 100 may be discarded.

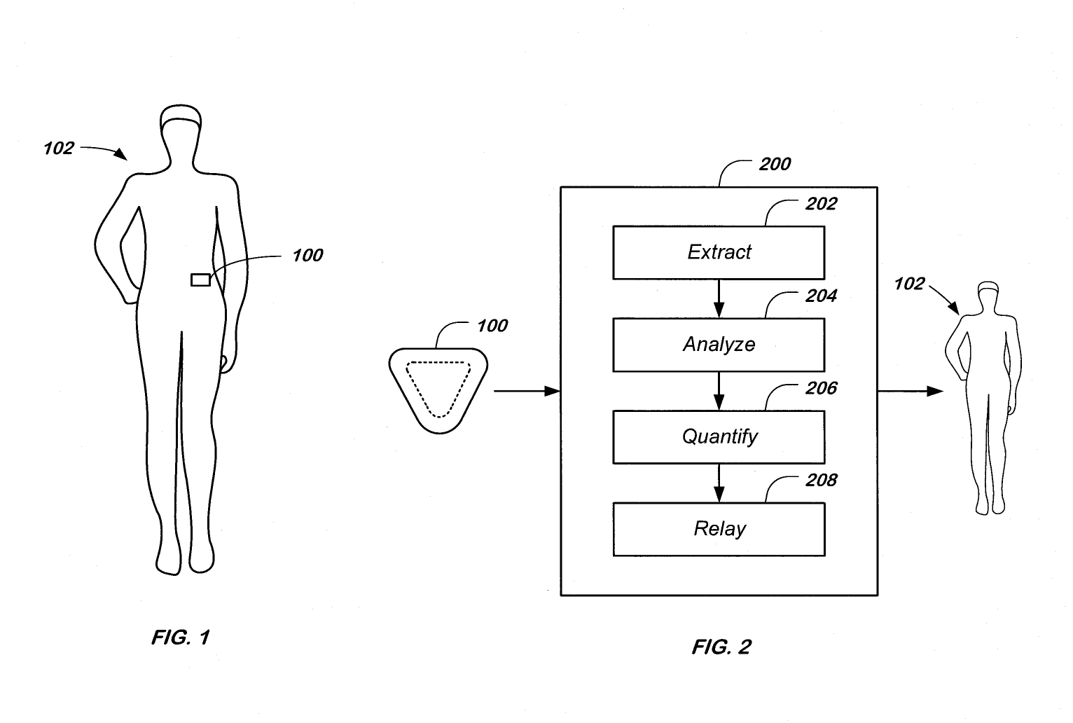

FIG. 2 illustrates one aspect of a processing facility 200 to process a biological sample collection device 100. The central processing facility 200 extracts 202 the biochemicals from the biological sample collection device 100, analyzes 204 the biochemicals, quantifies 206 them, and relays 208 that information to the subject 102 or a caregiver. As shown in FIG. 2, after the subject 102 has worn the biological sample collection device 100 for a prescribed period (e.g., from minutes to weeks, or other suitable period), the subject 102 returns the biological sample collection device 100 to a central processing facility 200 that converts the raw samples collected on the biological sample collection device 100 into a data form that the subject 102 can practically utilize. The advantage of this approach is that the central processing facility 200 does not have the same restrictions on cost, size, power, or chemical consumption that exist in the field.

The central processing facility 200 may use any suitable analytical procedures, such as a titration, for example, to analyze the analyte. In some aspects, conventional biochemical analysis techniques can be employed to extract and analyze the biological samples from the biological sample collection device 100. Such analytical techniques may include, for example, ELISA (enzyme linked immuno-assay), PCR (polymer chain reaction), FTIR (Fourier transform infrared spectroscopy), cell culture, mass spectroscopy, liquid chromatography, among other state of the art chemical analysis techniques. In one aspect, a reverse iontophoresis process may be used to drive the biochemicals/chemicals out of the collection device 100 and extract the analytes for analysis.

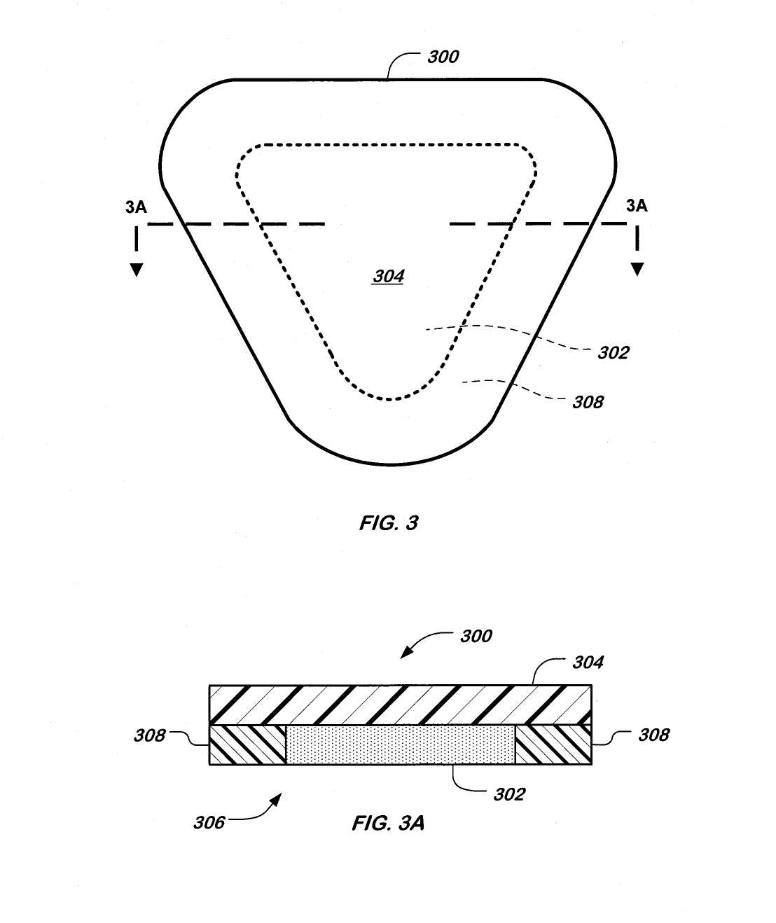

FIGS. 3-8A illustrate various biological sample collection devices that employ various techniques for collecting the biological samples from the subject 102 (FIGS. 1, 2). FIG. 3 illustrates one aspect of a biological sample collection device 300 that comprises an adsorbent material 302. In various aspects, the adsorbent material 302 adsorbs perspiration, blood, excreted oils, or other fluids secreted by the body of the subject 102.

FIG. 3A is a cross-sectional view of the biological sample collection device 300 shown in FIG. 3 taken along line 3A-3A. The biological sample collection device 300 comprises a top cover plate 304 (such as may be fabricated from a suitable polymeric material), which acts as a support structure for the biological sample collection device 300 and is placed facing away from the body of the subject 102 (FIGS. 1, 2). A bottom portion 306 attached to the top cover plate 304 comprises a biological sample collection portion to collect a biological sample from the body of the subject 102 and a fastening portion to affix the biological sample collection device 300 to the body of the subject 102. In one aspect, the biological sample collection portion comprises an adsorbent material 302, which is placed in contact with the body of the subject 102 such that perspiration, blood, excreted oils, or other fluids secreted by the body of the subject 102 can be absorbed by the adsorbent material 302. In one aspect, the biological sample collection device 300 can be affixed to the subject 102 by an adhesive portion 308 provided on the bottom portion 306. In other aspects, other techniques may be employed to affix the biological sample collection device 300 to the subject 102. The adsorbent material 302 and the adhesive portion 308 are affixed to the top cover plate 304 using any suitable fastener and/or bonding technique including, but not limited to, snaps, buttons, glue, weld, friction, adhesive, rivet, screw, press fitting, electrostatic bonding, ultrasonic welding, sowing. Other elements or components may be coupled to the top cover plate 304 in a similar manner.

The adsorption, atoms, ions, biomolecules or molecules of gas, liquid, or dissolved solids from the body of the subject 102 adhere to the surface of the adsorbent material 302. This process creates a film of the adsorbate (the molecules or atoms being accumulated) on the surface of the adsorbent material 302. It differs from absorption, in which a fluid permeates or is dissolved by a liquid or solid. Similar to surface tension, adsorption is a consequence of surface energy. In the adsorbent material 302, all the bonding requirements (be they ionic, covalent, or metallic) of the constituent atoms of the material are filled by other atoms in the material. Atoms on the surface of the adsorbent material 302, however, are not wholly surrounded by other adsorbent atoms and therefore can attract adsorbates from the body of the subject 102 such as perspiration, blood, excreted oils, or other fluids. The adsorption process generally may be classified as physisorption (characteristic of weak van der Waals forces) or chemisorption (characteristic of covalent bonding). It may also occur due to electrostatic attraction. The adsorbent material 302 may be formed using many natural physical, biological, or chemical materials such as activated charcoal, synthetic resins, nanoporous carbon. Adsorption, ion exchange, and chromatography are sorption processes in which certain adsorbates are selectively transferred from the fluid phase to the surface of insoluble, rigid particles suspended in a vessel or packed in a column.

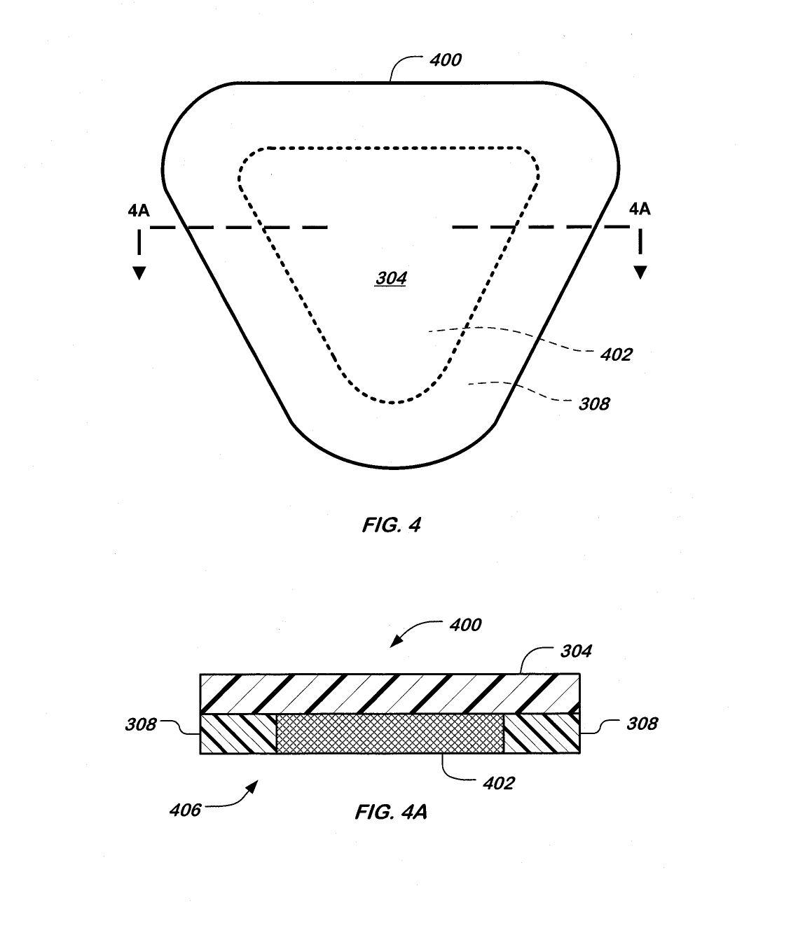

FIG. 4 illustrates one aspect of a biological sample collection device 400 that comprises an absorbent material 403. In various aspects, the absorbent material 403 comprises a polymer absorbent, zeolites, silica absorbents, or activated charcoal to absorb perspiration, blood, excreted oils, or other secretions from the body of the subject 102 (FIGS. 1, 2). In absorption, atoms, molecules, or ions enter some bulk phase—gas, liquid or solid material and the molecules are taken up by the volume of the absorbent material 402 of the absorbent material 402, not by the surface (as in the case for adsorption).

FIG. 4A is a cross-sectional view of the biological sample collection device 400 shown in FIG. 4 taken along line 4A-4A. The biological sample collection device 400 comprises a top cover plate 304, which acts as a support structure for the biological sample collection device 400 and is placed facing away from the body of the subject 102 (FIGS. 1, 2). A bottom portion 406 attached to the top cover plate 304 comprises a biological sample collection portion to collect a biological sample from the body of the subject 102 and a fastening portion to affix the biological sample collection device 400 to the body of the subject 102. In one aspect, the biological sample collection portion comprises an absorbent material 402, which is placed in contact with the body of the subject 102 such that perspiration, blood, excreted oils, or other fluids secreted by the body of the subject 102 can be absorbed by the volume of the absorbent material 402. In one aspect, the biological sample collection device 400 can be affixed to the subject 102 by an adhesive portion 308 provided on the bottom portion 406. In other aspects, other techniques may be employed to affix the biological sample collection device 400 to the subject 102. The adsorbent material 402 and the adhesive portion 308 are affixed to the top cover plate 304 using any suitable fastener and/or bonding technique including, but not limited to, snaps, buttons, glue, weld, friction, adhesive, rivet, screw, press fitting, electrostatic bonding, ultrasonic welding, sowing. Other elements or components may be coupled to the top cover plate 304 in a similar manner.

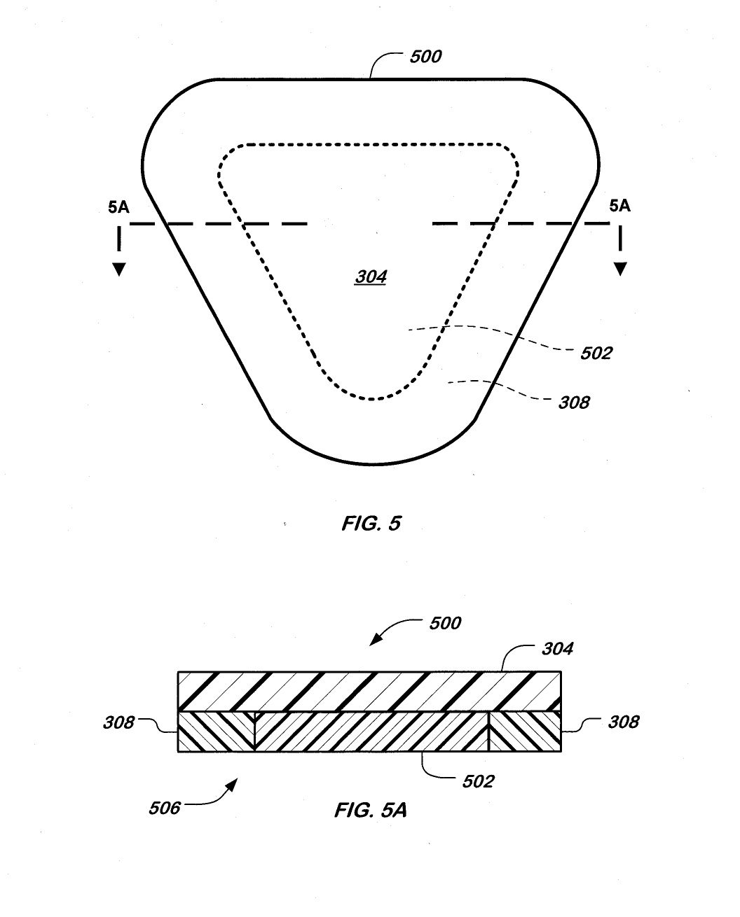

FIG. 5 illustrates one aspect of a biological sample collection device 500 that comprises an adhesive material 502. In various aspects, the adhesive material 502 retains skin cells or hair follicles when the biological sample collection device 500 is removed from the subject 102 (FIGS. 1, 2). The skin cells and hair follicles can be removed from the adhesive material 502 and then be subjected to genetic analysis.

FIG. 5A is a cross-sectional view of the biological sample collection device 500 shown in FIG. 5 taken along line 5A-5A. The biological sample collection device 500 comprises a top cover plate 304, which acts as a support structure for the biological sample collection device 500 and is placed facing away from the body of the subject 102 (FIGS. 1, 2). A bottom portion 506 attached to the top cover plate comprises a biological sample collection portion to collect a biological sample from the subject 102 and a fastening portion to affix the biological sample collection device 500 to the body of the subject 102. In one aspect, the biological sample collection portion comprises an adhesive material 402, which is placed in contact with the body of the subject 102 such that skin cells and hair follicles can adhere to the surface of the adhesive material 502. In one aspect, the biological sample collection device 500 can be affixed to the subject 102 by an adhesive portion 308 provided on the bottom portion 506. In another aspect, the biological sample collection device 500 may be affixed to the body of the subject 102 using just the adhesive portion 502 to obviate the need for a separate adhesive portion 308 for affixing purposes. Nevertheless, different adhesive portions 308, 502 having different adhesive properties may be provided in certain configurations. In other aspects, other techniques may be employed to affix the biological sample collection device 500 to the subject 102. The adhesive material 502 and the adhesive portion 308 are affixed to the top cover plate 304 using any suitable fastener and/or bonding technique including, but not limited to, snaps, buttons, glue, weld, friction, adhesive, rivet, screw, press fitting, electrostatic bonding, ultrasonic welding, sowing. Other elements or components may be coupled to the top cover plate 304 in a similar manner.

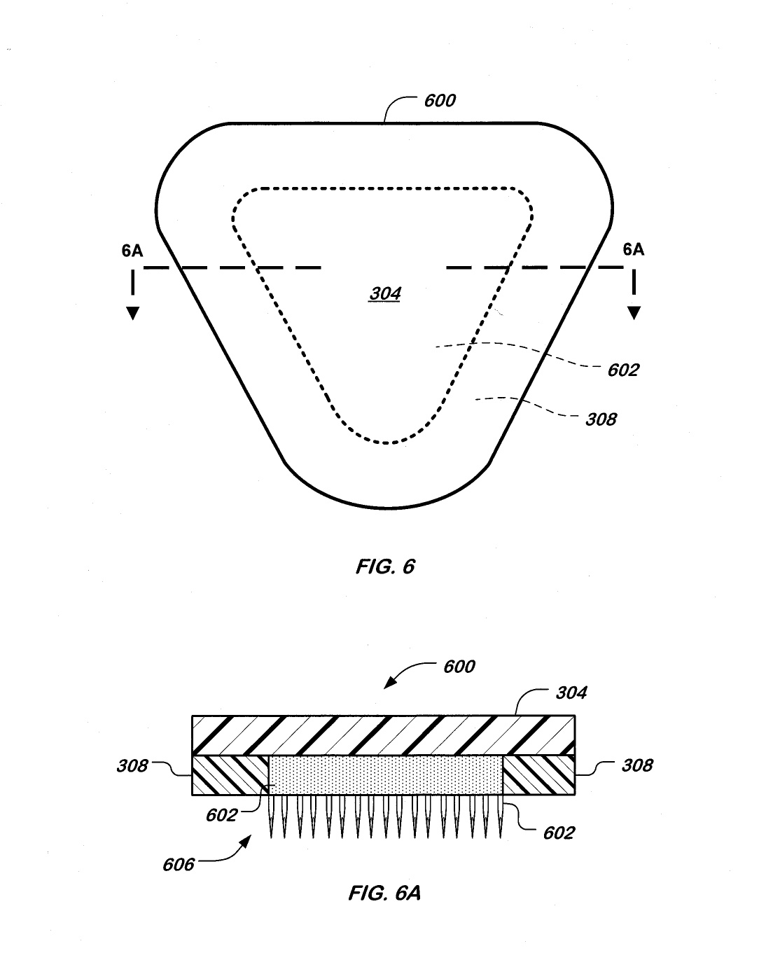

FIG. 6 illustrates one aspect of a biological sample collection device 600 that comprises at least one micro-needle 602. In one aspect, the at least one micro-needle 602 pierces the skin of the subject 102 (FIGS. 1, 2) and draws blood or extra-cellular fluid from the subject 102. The at least one micro-needle 602 is configured to puncture the stratum corneum layer of the skin and draw sub pore blood cells or extra cellular fluid from beneath the skin. The at least one micro-needle 602 may be formed of medical grade stainless steel or similar materials.

FIG. 6A is a cross-sectional view of the biological sample collection device 600 shown in FIG. 6 taken along line 6A-6A. The biological sample collection device 600 comprises a top cover plate 304, which acts as a support structure for the biological sample connection device 600 and is placed facing away from the body of the subject 102 (FIGS. 1, 2). A bottom portion 606 attached to the top cover plate 304 comprises a biological sample collection portion to collect a biological sample from the subject 102 and a fastening portion to affix the biological sample collection device 600 to the body of the subject 102. In one aspect, the biological sample collection portion comprises a sorbent material 604, which may be an adsorbent material 302 or an absorbent material 402 depending on the specific configuration, and a plurality of micro-needles 602 to draw blood or extra-cellular fluid from the subject 102. In one aspect, the biological sample collection device 600 can be affixed to the subject 102 by an adhesive portion 308 provided on the bottom portion 606. In other aspects, other techniques may be employed to affix the biological sample collection device 600 to the subject 102. The sorbent material 604 and the adhesive portion 308 are affixed to the top cover plate 304 using any suitable fastener and/or bonding technique including, but not limited to, snaps, buttons, glue, weld, friction, adhesive, rivet, screw, press fitting, electrostatic bonding, ultrasonic welding, sowing. The micro-needles 602 are embedded in the sorbent material 604 using any suitable technique. Other elements or components may be coupled to the top cover plate 304 in a similar manner.

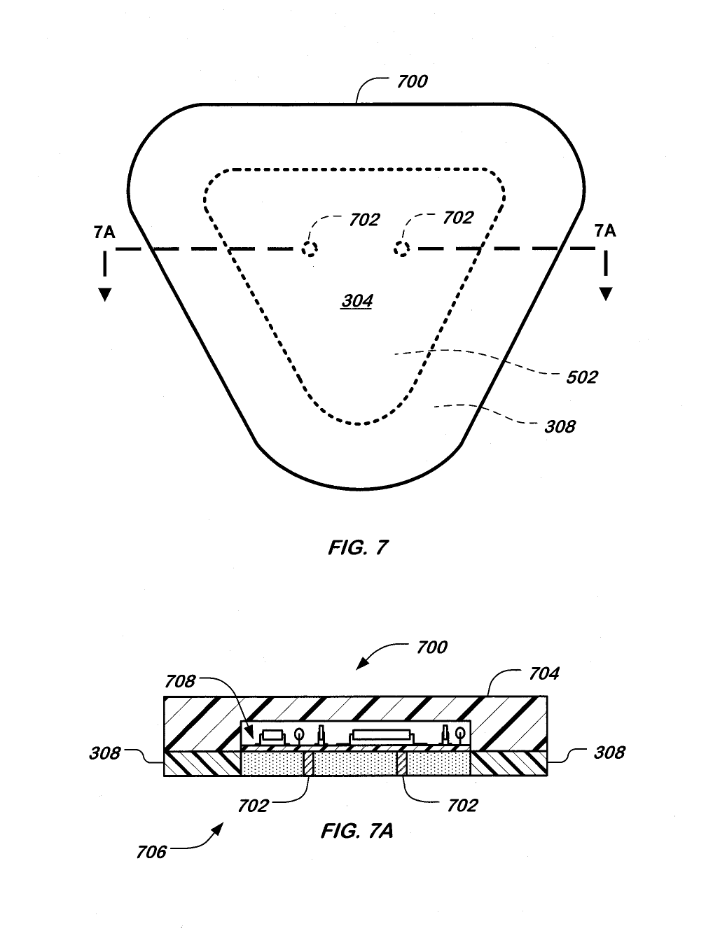

FIG. 7 illustrates one aspect of a biological sample collection device 700 that comprises electrodes 702 and an electronic module 708 comprising electronic components to extract body fluids from the subject 102 (FIGS. 1, 2) via reverse electrophoresis. In one aspect, the biological sample collection device 700 may include electrodes 702 to force analytes out through the skin using reverse iontophoresis. Those skilled in the art will appreciate that iontophoresis is used as a delivery method to drive materials (chemicals) from a patch into the bloodstream. Iontophoresis (a.k.a. Electromotive Drug Administration (EMDA)) is a technique using a small electric charge to deliver a medicine or other chemical through the skin. It is basically an injection without the needle. It is a non-invasive process of propelling high concentrations of a charged substance, normally a medication or bioactive agent, transdermally by repulsive electromotive force using a small electrical charge applied to an iontophoretic chamber containing a similarly charged active agent and its vehicle. In the biological sample collection device 700, however, the iontophoresis process is used in reverse to drive the biochemical/chemicals samples out of the skin of the subject 102 and into a sorbent material 710 on the biological sample collection device 700.

FIG. 7A is a cross-sectional view of the biological sample collection device 700 shown in FIG. 7 taken along line 7A-7A. The biological sample collection device 700 comprises a top cover plate 704 (such as may be fabricated from a suitable polymeric material), which acts as a support structure for the biological sample collection device 700 and is placed facing away from the body of the subject 102 (FIGS. 1, 2). A bottom portion 706 attached to the top cover plate 704 comprises a biological sample collection portion to collect a biological sample from the subject 102 and a fastening portion to affix the biological sample collection device 700 to the body of the subject 102. In one aspect, the biological sample collection portion comprises a sorbent material 710, which may be an adsorbent material 302 or an absorbent material 402 depending of the specific configuration, two electrodes 702, and electronic components 708 to force analytes out through the body of the subject 702 and into the sorbent material 710. The biological sample collection device 700 capable of reverse iontophoresis includes an on-board battery and the additional electronic components 708. In one aspect, the biological sample collection device 700 can be affixed to the subject 102 by an adhesive portion 308 provided on the bottom portion 706. In other aspects, other techniques may be employed to affix the biological sample collection device 700 to the subject 102. The sorbent material 710 and electronic components 708 and the adhesive portion 308 are affixed to the top cover plate 304 using any suitable fastener and/or bonding technique including, but not limited to, snaps, buttons, glue, weld, friction, adhesive, rivet, screw, press fitting, electrostatic bonding, ultrasonic welding, sowing. Other elements or components may be coupled to the top cover plate 304 in a similar manner.

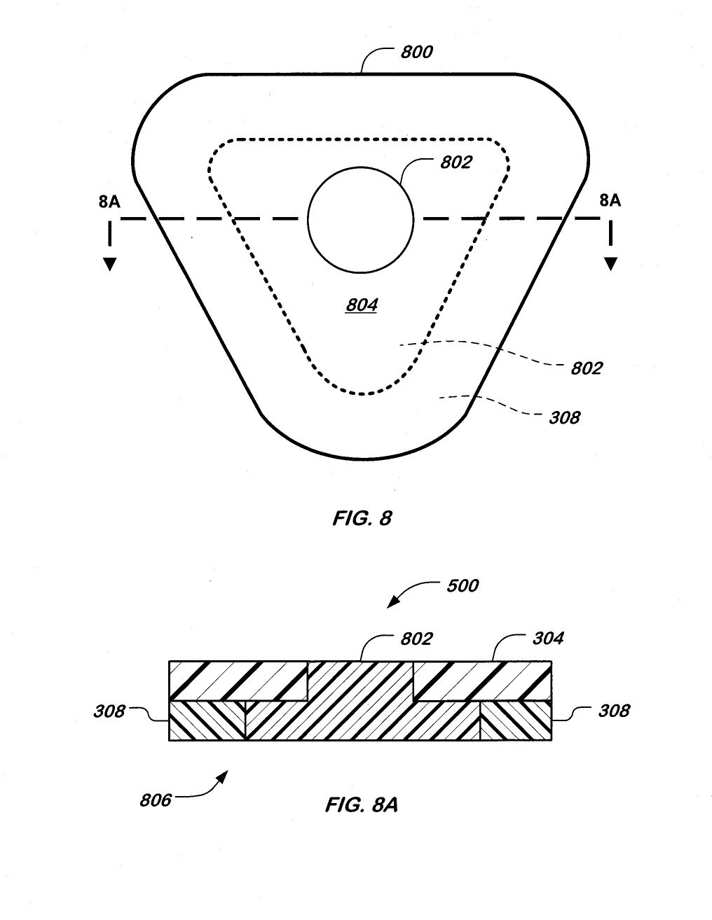

FIG. 8 illustrates one aspect of a biological sample collection device 800 for detecting the presence or absence of a substance using a color indicator 802. In other aspects, any of the biological sample collection devices 100, 300, 400, 500, 600, 700 described herein in reference to corresponding FIGS. 1 and 3-7 can be configured with the color indicator 802 to add the functionality for detecting the presence or absence of a substance. A precursor analysis may be done on the biological sample collection device 800 using a reactive material that will react to a certain physiological parameter or biomarker sample and provide a color indication in response thereto. The subject 102 (FIGS. 1, 2) then may take action in accordance with the indicated color. A different action may be taken based on the result such as the presence or absence of a particular color. For example, in accordance with one indication (e.g., a particular color change), the subject 102 may deliver the biological sample collection device 800 to the processing facility 200 and in accordance with another indication (e.g., no color change), the biological sample collection device 800 can be disposed of. The biological sample collection device 800 may comprise an analyzer assay similar to a home pregnancy test, or calorimetric test, such that the assay can be included in the biological sample collection device 800. In one aspect, when the color is red the subject 102 may be instructed to seek medical attention immediately, when the color is yellow the subject 102 may be instructed to mail the biological sample collection device 100 to the processing facility 200, or dispose the biological sample collection device 100 when the color is green or colorless. Any suitable color combination, or lack of color, can be used to notify the subject 102 of the particular physiological parameter or biomarker.

FIG. 8A is a cross-sectional view of the biological sample collection device 800 shown in FIG. 8 taken along line 8A-8A. The biological sample collection device 800 comprises a top cover plate 804 (such as may be fabricated from a suitable polymeric material), which acts as a support structure for the biological sample to collection device 800 and is placed facing away from the body of the subject 102 (FIGS. 1, 2). A bottom portion 806 attached to the top cover plate 804 comprises a biological sample collection portion to collect a biological sample from the subject 102 and a fastening portion 308 provided on the bottom portion 806 to affix the biological sample collection device 300 to the body of the subject 102. In one aspect, the biological sample collection portion material 802 comprises an analyzer assay embedded therein that changes color based on the presence or absence of a substance secreted by the subject 102. In one aspect, the biological sample collection device 800 can be affixed to the subject 102 by an adhesive portion 308. In other aspects, other techniques may be employed to affix the biological sample collection device 800 to the subject 102.

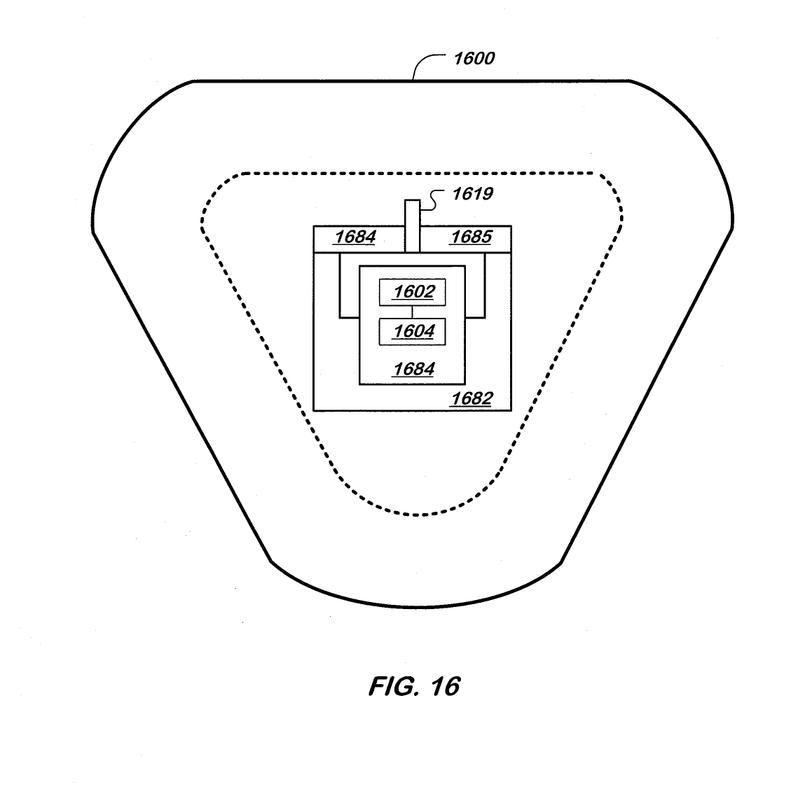

FIG. 16 illustrates one aspect of a biological sample collection device 1600 comprising a framework for generating an operational voltage, a real time clock 1602, and a memory 1604. The real time clock 1602 and the memory 1604 may be coupled to the top cover plate or other suitable structure within the sample collection device 1600 capable of supporting real time clock 1602 and memory 1604. In this context coupled refers to the real time clock 1602 and memory 1604 being attached to, embedded in, located within, or otherwise supported by the structure of the top cover plate. The framework for generating an operational voltage, the real time clock 1602, and the memory 1604 may be coupled to the top cover plate of the biological sample collection device 1600 using any suitable fastener and/or bonding technique including, but not limited to, snaps, buttons, glue, weld, friction, adhesive, rivet, screw, press fitting, electrostatic bonding, ultrasonic welding, sowing. Other elements or components may be coupled to the top cover plate 304 in a similar manner.

The real time clock 1602 and the memory 1604 record or time-stamp (e.g., record in memory the date and/or time) when the biological sample collection device is applied and/or removed from the body of the subject 102 (FIGS. 1 and 2). In various aspects, the biological sample collection device 1600 may comprise any or all of the features of the biological sample collection devices 100, 300, 400, 500, 600, 700, 800 described in connection with respective FIGS. 1 and 3-8. In one aspect, the biological sample collection device 1600 may comprise on onboard sensing mechanism 1606 to detect the application and/or removal of the biological sample collection device 1600 from the body of the subject 102. In one aspect, the real time clock 1602 and the memory 1604 do not require an on-board battery but may incorporate a framework 1682 for generating an operational voltage similar to framework used in an ingestible event marker (IEM) by Proteus Biomedical, Inc. of Redwood City, Calif. In other aspects, the biological sample collection device 1600 may comprise a small battery suitable to activate the real time clock 1602 and enable the memory 1604 to record the time-stamp. In other aspects, the biological sample collection device 1602 detects when it is applied to the body of the subject 102 and records a time-stamp at that time. In such aspects, the processing facility 200 (FIG. 2) records a time-stamp when it receives the biological sample collection device 1600.

In one aspect, the framework 1682 of the biological sample collection device 1600 provides a chassis for attaching, depositing upon, or securing multiple components. In one aspect, a material 1684 is physically associated with the framework 1682. The material 1684 may be chemically deposited on, evaporated onto, secured to, or built-up on the framework all of which may be referred to herein as “deposit” with respect to the framework 1682. The material 1684 is deposited on one side of the framework 1682. The materials of interest that can be used as material 1684 include, but are not limited to: Cu or Cul. The material 1684 is deposited by physical vapor deposition, electrodeposition, or plasma deposition, among other protocols. The material 1684 may be from about 0.05 to about 500 μm thick, such as from about 5 to about 100 μm thick. The shape is controlled by shadow mask deposition, or photolithography and etching. Additionally, even though only one region is shown for depositing the material, each of the biological sample devices 1600 may contain two or more electrically unique regions where the material 1684 may be deposited, as desired.

At a different portion of the same side of the framework 1682, another material 1686 is deposited, such that the first and second materials 1684 and 1686 are dissimilar and are separated by a non-conducting skirt 1619. More specifically, the first and second materials 1684 and 1686 are selected such that they form a voltage potential difference when in contact with a conducting liquid, such as body fluids secreted by the body of subject 102, for example. Thus, when the biological sample devices 1600 is in contact with and/or partially in contact with a conducting liquid on the surface of the body of the subject 102, a current path 1692 is formed through the conducting liquid between first and second material 1684 and 1686. Although not shown, in one aspect, the second material 1686 may be located opposite to the first material 1684. The scope of the present disclosure is not limited by the side selected and the term “different side” can mean any of the multiple sides that are different from the first selected side. Furthermore, although the shape of the system is shown as a square, the shape may be any geometrically suitable shape. The materials 1684 and 1686 are selected such that they produce a voltage potential difference when the biological collection device 1600 is in contact with a conducting liquid, such as body fluid, on the surface of the body of the subject 102 when the biological collection device 1600 is applied to the body of the subject 102. The materials of interest for material 1686 include, but are not limited to: Mg, Zn, or other electronegative metals. As indicated above with respect to the material 1684, the material 1686 may be chemically deposited on, evaporated onto, secured to, or built-up on the framework. Also, an adhesion layer may be necessary to help the material 1686 (as well as material 1684 when needed) to adhere to the framework 1682. Typical adhesion layers for the material 1686 are Ti, TiW, Cr or similar material. Anode material and the adhesion layer may be deposited by physical vapor deposition, electrodeposition or plasma deposition. The material 1686 may be from about 0.05 to about 500 μm thick, such as from about 5 to about 100 μm thick. However, the scope of the present disclosure is not limited by the thickness of any of the materials nor by the type of process used to deposit or secure the materials to the framework 1682.

According to the disclosure set forth, the materials 1684 and 1686 can be any pair of materials with different electrochemical potentials. Additionally, in the aspects wherein the system 1680 is used in-vivo, the materials 1684 and 1686 may absorbable by the body of the subject 102. More specifically, the materials 1684 and 1686 can be made of any two materials appropriate for the environment in which the biological collection device 1600 will be operating. For example, when the biological collection device 1600 is in contact with an ionic solution, such as perspiration. Suitable materials are not restricted to metals, and in certain aspects the paired materials are chosen from metals and non-metals, e.g., a pair made up of a metal (such as Mg) and a salt (such as CuCl or Cul). With respect to the active electrode materials, any pairing of substances—metals, salts, or intercalation compounds—with suitably different electrochemical potentials (voltage) and low interfacial resistance are suitable.

Materials and pairings of interest include, but are not limited to, those reported in TABLE 1 below. In one aspect, one or both of the metals may be doped with a non-metal, e.g., to enhance the voltage potential created between the materials as they come into contact with a conducting liquid. Non-metals that may be used as doping agents in certain aspects include, but are not limited to: sulfur, iodine, and the like. In another aspect, the materials are copper iodine (Cul) as the anode and magnesium (Mg) as the cathode. Aspects of the present disclosure use electrode materials that are not harmful to the human body.

TABLE 1

| | Anode | Cathode |
|---|---|---|
| Metals | Magnesium, Zinc | |
| | Sodium (†), Lithium (†) | |
| | Iron | |
| Salts | | Copper salts: iodide, chloride, |
| | | bromide, sulfate, formate, (other |
| | | anions possible) |
| | | Fe³⁺ salts: e.g. orthophosphate, |
| | | pyrophosphate, (other anions |
| | | possible) |
| | | Oxygen (††) on platinum, gold |
| | | or other catalytic surfaces |
| Intercalation | Graphite with Li, K, Ca, | Vanadium oxide Manganese |
| compounds | Na, Mg | oxide |

Thus, when the biological collection device 1600 is in contact with a conducting fluid, a current path is formed through the conducting fluid between material 1684 and 1686. A control device 1688 is secured to the framework 1682 and electrically coupled to the materials 1684 and 1686. In one aspect, the control device 1688 includes electronic circuitry, for example the real time clock 1602 and memory 1604 capable of recording a time-stamp when the biological collection device 1600 is applied to the body of the subject 102 and the a control voltage is generated by the framework 1682. In other aspects, the control device 1688 may comprise logic is capable of performing additional functionality such as controlling and altering the conductance between the materials 1684 and 1686.

The voltage potential created between the materials 1684 and 1686 provides the power for operating the system as well as produces the current flow through the conducting fluid and the biological collection device 1600. In one aspect, the biological collection device 1600 operates in direct current mode. In an alternative aspect, the biological collection device 1600 controls the direction of the current so that the direction of current is reversed in a cyclic manner, similar to alternating current. As the conducting fluid or the electrolyte, where the fluid or electrolyte component is provided by a physiological fluid, e.g., perspiration, reaches the two materials 1684 and 1686 the path for current flow between the materials 1684 and 1686 is completed and, in one aspect, the current path through may be controlled by the control device 1688. Completion of the current path allows for the current to flow and in turn activate the real time clock 1602 and memory 1604 to record a time-stamp when the biological collection device 1600 is applied to the body of the subject 102.

In one aspect, the two materials 1684 and 1686 are similar in function to the two electrodes needed for a direct current power source, such as a battery. The conducting liquid acts as the electrolyte needed to complete the power source. The completed power source described is defined by the physical chemical reaction between the materials 1684 and 1686 of the biological collection device 1600 and the surrounding fluids of the body. The completed power source may be viewed as a power source that exploits reverse electrolysis in an ionic or a conduction solution such as gastric fluid, blood, or other bodily fluids and some tissues. Additionally, the environment may be something other than a body and the liquid may be any conducting liquid. For example, the conducting fluid may be salt water or a metallic based paint.

In certain aspects, the two materials 1684 and 1686 may be shielded from the surrounding environment by an additional layer of material. Accordingly, when the shield is dissolved and the two dissimilar materials are exposed to the target site, a voltage potential is generated.

In certain aspects, the complete power source or supply is one that is made up of active electrode materials, electrolytes, and inactive materials, such as current collectors, packaging. The active materials are any pair of materials with different electrochemical potentials. Suitable materials are not restricted to metals, and in certain aspects the paired materials are chosen from metals and non-metals, e.g., a pair made up of a metal (such as Mg) and a salt (such as Cul). With respect to the active electrode materials, any pairing of substances—metals, salts, or intercalation compounds—with suitably different electrochemical potentials (voltage) and low interfacial resistance are suitable.

A variety of different materials may be employed as the materials that form the electrodes. In certain aspects, electrode materials are chosen to provide for a voltage upon contact with the target physiological site, e.g., the outer skin of the subject 102, sufficient to drive the power source of the biological collection device 1600. In certain aspects, the voltage provided by the electrode materials upon contact of the metals of the power source with the target physiological site is 0.001 V or higher, including 0.01 V or higher, such as 0.1 V or higher, e.g., 0.3 V or higher, including 0.5 volts or higher, and including 1.0 volts or higher, where in certain aspects, the voltage ranges from about 0.001 to about 10 volts, such as from about 0.01 to about 10 V.

In various aspects, the biological sample collection devices 100, 300, 400, 500, 600, 700, 800, 1600 described in connection with respective FIGS. 1, 3-8, and 16 may be used in combination with a transbody conductive communication module (e.g., a communication module that receives communications from the IEM, smart medical device or body-associated device). Additionally, in other aspects, the biological sample collection devices 100, 300, 400, 500, 600, 700, 800, 1600 may be used in combination with a physiological sensing module. Such biological sample collection systems (e.g., combination of a biological sample collection device with a transbody conductive module, or a sensing module, or both) may be configured to receive a transbody conductive communications (such as communications from an IEM, smart medical device or body-associated device) using a detection protocol. Transbody conductive communication detection protocols are processes in which the receiver is in a state in which it can receive communications from an IEM or smart parenteral device, and process the communications as desired, e.g., by performing one or more tasks, such as decoding the communication, storing the communication, time-stamping the communication, and re-communicating the communication, as described in greater detail below. Such combination biological sample collection devices also may be configured to perform a physiological data detection protocol when present in an active state, e.g., to obtain ECG data, accelerometer data, temperature data, as described in greater detail below.

Various aspects of biological sample collection systems provide a method of connecting medication that a subject 102 takes daily to the Internet and to the communication device (e.g., telephone or wireless communication device such as a cell phone or smart phone) of the subject 102. The IEM is generally configured as a pill taken by the subject 102. The IEM comprises an integrated circuit microchip and/or sensors embedded on or in the medication capsule or tablet (pill). The microchip and its sensors are powered up when wetted, which generally occurs when the pill is ingested. When ingested, the microchip starts generating physiologic communications that propagate through the body, like an electrocardiogram (EKG) or electromyography (EMG), and are communicated through the body. Like an EKG, the physiologic communications can be picked up by a receiver located on the subject 102. Most often the receiver is located on a wearable patch stuck on the torso of the subject 102. The patch has electrodes which detect the physiologic communications coming from the pill, measures heart rate and other parameters, and includes an accelerometer to measure body angle and activity level. From those measurements, processing devices on the patch can derive other metrics like amount of exercise, step count, how much sleep the subject gets at night. The receiver also includes a radio that uplinks the subject information (data) to the Internet usually via the communication device of the subject 102. The information associated with the subject 102 is then routed to servers and sent back to the subject 102 via the communication device. The subject 102 can then see what medications they took, when they took them, how that compares to what they were prescribed, and can also see metrics of how well they are doing. The subject 102 also can choose to share that information with professional caregivers such as doctors, nurses, family caregivers, joint social networks of people that have similar conditions so they can compare notes on how they are feeling, what medications work for them, what strategies work to help to keep their health improving, and so on.

In addition to receiving a conductive form of communication, such as one communicated by an identifier of an ingestible event marker, the receiver may further include one or more distinct physiological sensing modules having physiological parameter sensing abilities. The physiological sensing module may be implemented to sense various physiological parameters or biomarkers, such as, but not limited to: cardio-data, including heart rate, electrocardiogram (ECG), and the like; respiration rate, temperature; pressure; chemical composition of fluid, analyte detection in blood, fluid state, blood flow rate, accelerometer motion data. Where the receiver has physiological parameter or biomarker sensing capability, the number of distinct parameters or biomarkers that the receiver may sense may vary, e.g., 1 or more, 2 or more, 3 or more, 4 or more, 5 or more, 10 or more. The term “biomarker” refers to an anatomic, physiologic, biochemical, molecular or other parameter associated with the presence and severity of specific disease states. Biomarkers are detectable and measurable by a variety of methods including physical examination, laboratory assays and medical imaging. Depending on the particular aspect, the receiver may accomplish one or more of these sensing functions using the communication receiving element, e.g., using electrodes of the receiver for receiving communications and sensing applications, or the receiver may include one or more distinct sensing elements that are different from the communication receiving element. The number of distinct sensing elements that may be present on (or at least coupled to) the receiver may vary, and may be one or more, two or more, three or more, four or more, five or more, ten or more.

In certain aspects, the receiver includes a set of two or more, such as two or three, electrodes that provide for dual functions of receiving communications and sensing. For example, in addition to receiving communications, the electrodes can also serve additional sensing functions. In certain aspects, the electrodes are used to generate electrocardiogram data. From that data, there are many kinds of processing that can be done, e.g., to detect various cardiac events, such as tachycardia, fibrillations, heart rate. The obtained electrocardiogram data can be used to titrate medications, or be used for alerts when an important change or significant abnormality in the heart rate or rhythm is detected. This data is also helpful in certain aspects for monitoring heart rate in patients who do not have pacemakers or as an alternative to patients who might normally require a Holter monitor or a Cardiac Event Monitor, portable devices for continuously monitoring the electrical activity of the heart for 24 hours or other devices. An extended recording period is useful for observing occasional cardiac arrthymias that are difficult to identify in shorter time periods.

In various aspects, the biological sample collection systems may comprise sensors, electronic recording devices, memory, communication components, an on-board battery to supply electrical power to the active components, a real time clock to time-stamp the time when the data collection is actually performed, one or more physiological parameter or biomarker sensing and recording abilities in combination with the physiological parameter or biomarker collection abilities such as, without limitation, cardio-data, including heart rate, electrocardiogram (ECG), and the like; respiration rate, temperature; pressure; chemical composition of fluid, e.g., analyte in blood, fluid state, blood flow rate, accelerometer motion data. The data can be stored in memory and when the biological sample collection device is sent to the processing facility 200 the data can be downloaded along with the biomarker.

Biological sample collection systems comprising receivers, configured with passive elements to collect physiological parameters or biomarkers for later analyses at a processing facility and a physiological data detection protocol when present in an active state, e.g., to obtain ECG data, accelerometer data, temperature data, will now be described in greater detail below.

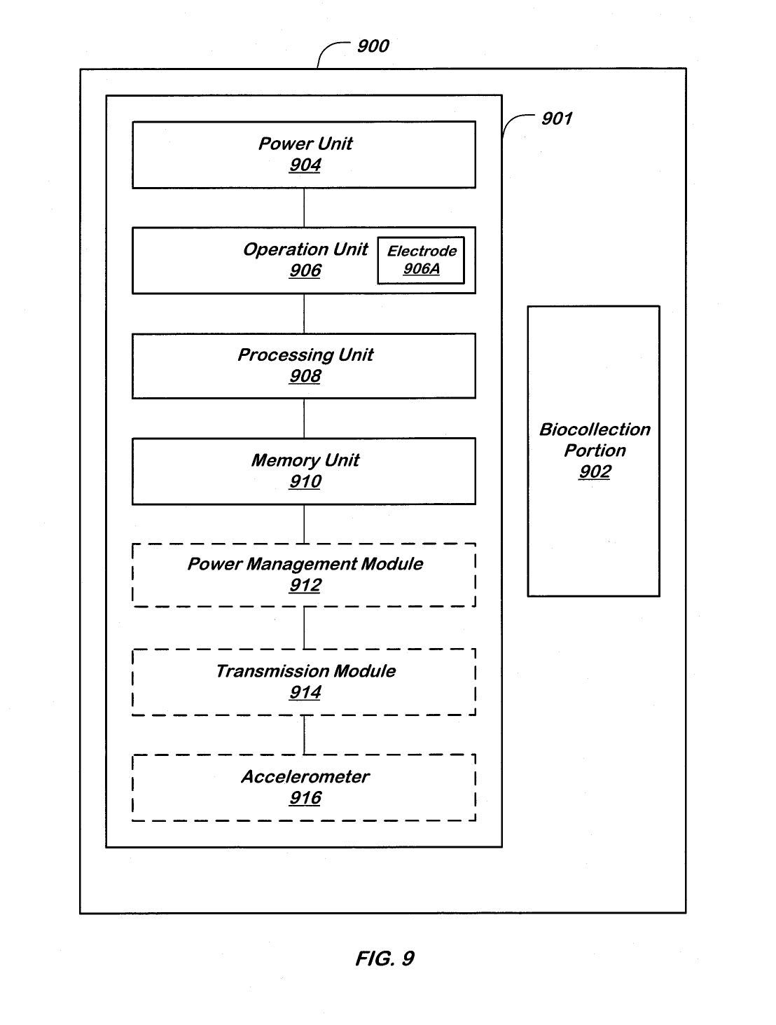

FIG. 9 illustrates one aspect of a biological sample collection system 900 comprising a receiver 901 portion and a biological sample collection portion 902. The biological sample collection portion 902 may comprise features, in any suitable configuration and combination, similar to any of the features of the biological sample collection devices 100, 300, 400, 500, 600, 700, 800, 1600 described in connection with respective FIGS. 1, 3-8, and 16. In one aspect, the receiver 901 includes a power unit 904, an operation unit 906 that includes an electrode 906A, an operation or processing unit 908, and a memory unit 910. The receiver 901 also includes a power management module 912 that controls the power consumption. The receiver 901 is capable of communicating with other near-by devices using a transmission module 914. Furthermore, the receiver 901 may include various features such as an accelerometer 916 for detection of the orientation of the receiver 901. In instances where the subject 102 (FIGS. 1, 2) is lying down or in a horizontal position, the receiver 901 is capable of detecting that position and the duration of time that the subject 102 remains in that position.

Additionally, the receiver 901 may further include one or more distinct physiological parameter sensing abilities. By physiological parameter sensing ability is meant a capability of sensing a physiological parameter or biomarker, such as, but not limited to: heart rate, respiration rate, temperature, pressure, chemical composition of fluid, e.g., analyte detection in blood, fluid state, blood flow rate, accelerometer motion data, IEGM (intra cardiac electrogram) data.

In accordance with the teaching of the present disclosure, the receiver aspects of the receiver 901 may be configured to receive communications. The communications may be in the form of conductively modulated information communicated by any physiologic part of the body or from a device that communicates by way of conduction through a body using ionic emission through controlled release of mass from solid into a conducting solution or fluid. The communication may be produced by an ionic emission module or an IEM or a smart-parenteral delivery system. Ingestible event markers of interest include those described in PCT Application Serial No. PCT/US2006/016370 published as WO/2006/116718; PCT Application Serial No. PCT/US2007/082563 published as WO/2008/052136; PCT Application Serial No. PCT/US2007/024225 published as WO/2008/063626; PCT Application Serial No. PCT/US2007/022257 published as WO/2008/066617; PCT Application Serial No. PCT/US2008/052845 published as WO/2008/095183; PCT Application Serial No. PCT/US2008/053999 published as WO/2008/101107; PCT Application Serial No. PCT/US2008/056296 published as WO/2008/112577; PCT Application Serial No. PCT/US2008/056299 published as WO/2008/112578; and PCT Application Serial No. PCT/US2008/077753 published as WO 2009/042812; the disclosures of which applications are herein incorporated by reference. Smart parenteral delivery systems are described in PCT Application Serial No. PCT/US2007/015547 published as WO 2008/008281; each of the foregoing disclosures is herein incorporated by reference in its entirety.

As the receiver 901 of these aspects is configured to receive data encoded in current flow through a conductive fluid, the receiver 901 and the device that emits the communication (such as an IEM) use the living body with which they are associated as a communication medium. To employ the body as a communication medium, the body fluids act as the conducting fluid and the body of the subject is used as a conduction medium for communication. As such, the communication transferred between ionic emission device and any other emitting device and the receiver, such as the receiver 901, travels through the body of the subject 102. The conductively communicated information of interest may be communicated through and received from the skin and other body tissues of the subject body in the form of electrical alternating current (a.c.) communications that are conducted through the body tissues. As a result, such receivers do not require any additional cable or hard wire connection between the communicating device and the receiver.

As the receivers 901 are configured to receive conductively communicated information, they may include a transbody conductive communication module. The transbody conductive communication module is a functional module that is configured to receive a conductive communication, such as a communication emitted by an IEM. Where desired, the transbody conductive communication module may be implemented by a high power functional block. In some instances, the communication by the transbody conductive communication module is configured to receive is encoded information, by which is meant that the information has been modulated in some manner (for example using a protocol such as binary phase shift keying (BPSK), frequency shift keying (FSK), amplitude shift keying (ASK)). In such instances, the receivers and transbody conductive communication module thereof are configured to decode encoded information, such as information communicated by an IEM. The receivers may be configured to decode the encoded in a low signal to noise ratio (SNR) environment, e.g., where there may be substantial noise in addition to the information of interest, e.g., an environment having an SNR of 7.7 dB or less. The receivers may be further configured to decode the encoded information with substantially no error. In certain aspects, the receiver has a high coding gain, e.g., a coding gain ranging from 6 dB to 12 dB, such as a coding gain ranging from 8 dB to 10 dB, including a coding gain of 9 dB. The receivers of aspects disclosed herein can decode encoded information with substantially no error, e.g., with 10% error or less.

In those aspects where the received information is encoded, such as where the received information is an encoded IEM communication, the transbody conductive communication module may be configured to process the received communication with at least one demodulation protocol, where the transbody conductive communication module may be configured to process the received communication with two or more, three or more, four or more, different demodulation protocols, as desired. When two or more different demodulation protocols are employed to process a given encoded information, the protocols may be run simultaneously or sequentially, as desired. The received information may be processed using any convenient demodulation protocol. Demodulation protocols of interest include, but are not limited to: Costas Loop demodulation (for example, as described in PCT Application Serial No. PCT/US07/024,225 and published as WO 2008/063626, the disclosure of which is herein incorporated by reference); coherent demodulation (for example, as described in PCT Application Serial No. PCT/US07/024,225 and published as WO 2008/063626, the disclosure of which is herein incorporated by reference); accurate, low overhead iterative demodulation (for example, as described in PCT Application Serial No. PCT/US07/024,225 and published as WO 2008/063626, the disclosure of which is herein incorporated by reference); incoherent demodulation; and differential coherent demodulation.

In some instances, a coherent demodulation protocol is employed. Coherent demodulation modules that may be employed in aspects of the receivers include, but are not limited to, those described in PCT Application Serial No. PCT/US2007/024225; the disclosure of which is herein incorporated by reference.

In some instances, a differential coherent demodulation protocol is employed. Differentially coherent demodulation compares the phase of adjacent bits in a Binary phase-shift keying modulated communication (BPSK). For example an 8 bit binary code of 11001010 would result in a differential series of bits 0101111. Since the technique leverages phase differences between adjacent bits, it is inherently more robust against frequency instability and drift than a coherent demodulation scheme.

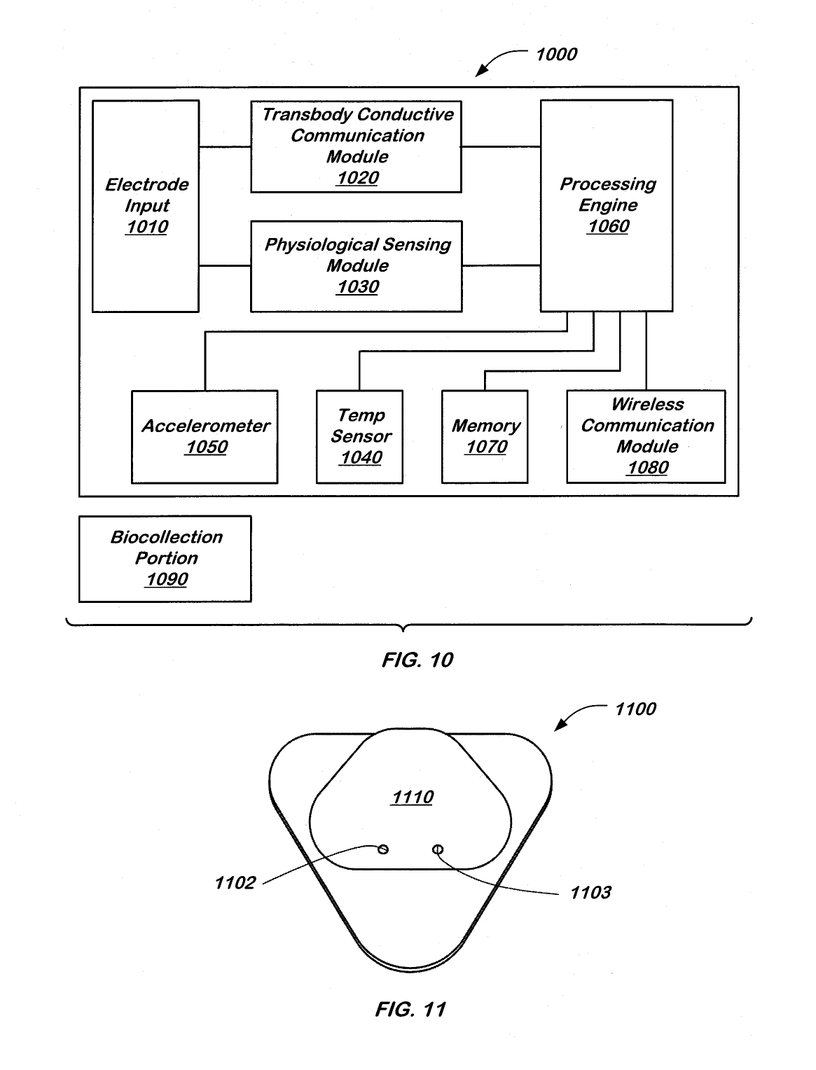

FIG. 10 is a block functional diagram of one aspect of an integrated circuit component of a receiver 1000 component of the biological sample collection system 900 shown in FIG. 9. In FIG. 10, the receiver 1000 includes an electrode input 1010. Electrically coupled to the electrode input 1010 are a transbody conductive communication module 1020 and a physiological sensing module 1030. In one aspect, the transbody conductive communication module 1020 is implemented as a first frequency (e.g., high frequency (HF)) chain and the physiological sensing module 1030 is implemented as a second frequency (e.g., low frequency (LF)) chain. Also shown are CMOS temperature sensing module 1040 (for detecting ambient temperature) and a 3-axis accelerometer 1050. The receiver 1000 also includes a processing engine 1060 (for example, a microcontroller and digital signal processor), a non-volatile memory 1070 (for data storage), and a wireless communication module 1080 (for data transmission to another device, for example in a data upload action). A biological sample collection portion 1090 may be provided in combination with the receiver 1000. In various aspects, the biological sample collection portion 1090 comprises features, in any suitable configuration and combination, similar to any of the features of the biological sample collection devices 100, 300, 400, 500, 600, 700, 800, 1600 described in connection with respective FIGS. 1, 3-8, and 16.

FIG. 11 illustrates one aspect of an external biological sample collection system 1100 comprising a receiver portion and a biological sample collection portion. FIG. 11 shows one aspect of a combined biological sample collection system 1100 that is configured to be placed on an external topical location of a subject 102 (FIGS. 1, 2), such as a chest area. The receiver includes an upper housing plate 1110 (such as may be fabricated from a suitable polymeric material), and includes a manually depressible operation button 1102 and a status identifier LED 1103, which may be used to relay to an observer that the receiver is operating. Manually depressible operation button 1102 can be manually manipulated to transition the receiver from a storage mode to a non-storage mode. When the receiver is in the storage mode, a micro-controller of the receiver may remain in a low duty cycle active state at all times to process input from the on/off button, and the digital signal processor (DSP) of the receiver powered off. When the on/off button is depressed to turn on the receiver, the micro-controller de-bounces the input and powers the DSP into its idle state. While in storage mode, the device may draw less than 10 μA, including 5 μA of current or less, such as 1 μA or less and including 0.1 μA or less. This configuration enables the device to remain at greater than 90% useful battery life if stored for one month (assuming the presence of a 250 mAH battery). Such a button may also be employed for other functions. For example, such a button may be employed to instruct the receiver to obtain certain types of data. In addition or alternatively, such a button may be employed to manually instruct the receiver to transfer data to another device.

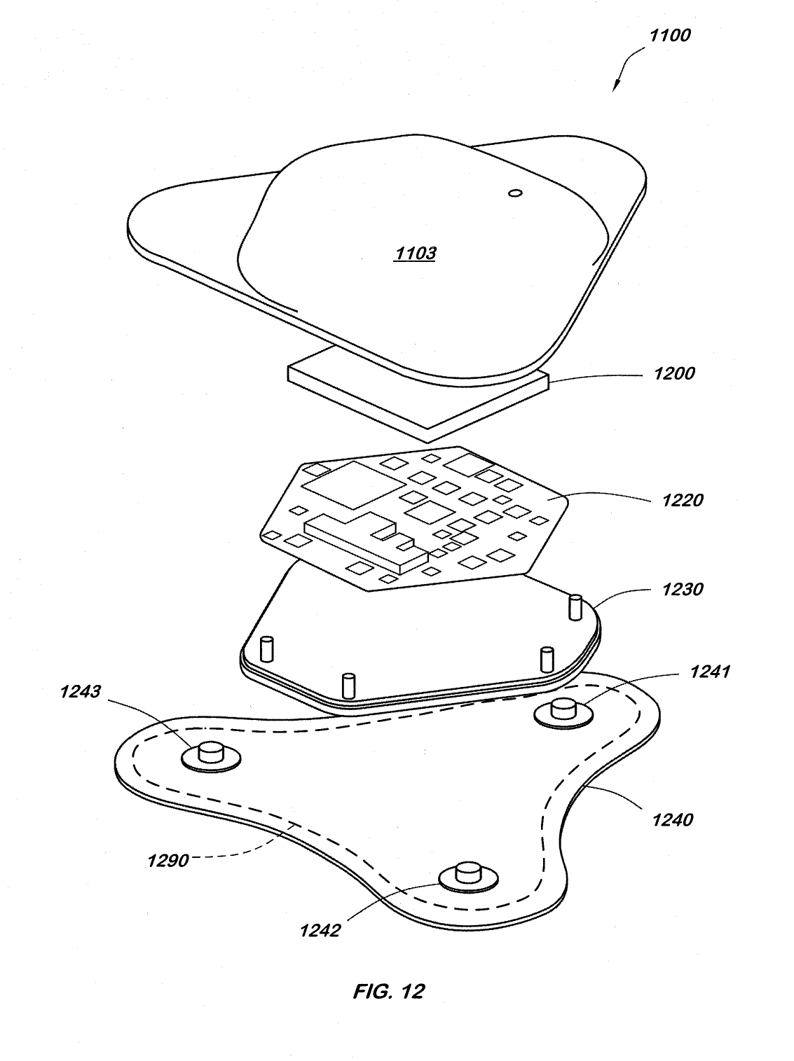

FIG. 12 is an exploded view of the biological sample collection system 1100 shown in FIG. 11. As shown in FIG. 12, the combined biological sample collection system 1100 includes the upper housing plate 1110, a rechargeable battery 1200, an integrated circuit component 1220, and a bottom housing plate 1230. The bottom housing plate 1230 snap fits into the top housing plate 1110 to seal the battery 1200 and the integrated circuit component 1220 in a fluid tight housing. While a snap-fit interaction is illustrated, any convenient mating scheme may be employed, such that the top and bottom housing plates may interact via inter-locking grooves, may be held together via a suitable adhesive, may be welded together. In some instances, the electrical components may be molded into the top and/or bottom housing plates. Also shown is adhesive patch 1240 which snaps into top housing plate 1110 and includes conductive studs 1241 to 1243, which studs serve as electrode contacts with the body during receiver use. The adhesive patch 1240 comprises a biological sample collection module 1290, which may comprise features, in any suitable configuration and combination, similar to any one of the features of the biological sample collection devices 100, 300, 400, 500, 600, 700, 800, 1600 described in connection with respective FIGS. 1, 3-8, and 16. The conductive studs 1241 to 1243 are in electrical contact with the integrated circuit component 1220, e.g., via wires or other conductive members associated with the upper housing plate 1110. In one aspect, the upper housing plate 1110 includes conductive members configured to receive the conductive studs 1241 to 1243 coupled to wires (not shown) which in turn provide electrical connection to the integrated circuit component 1220.

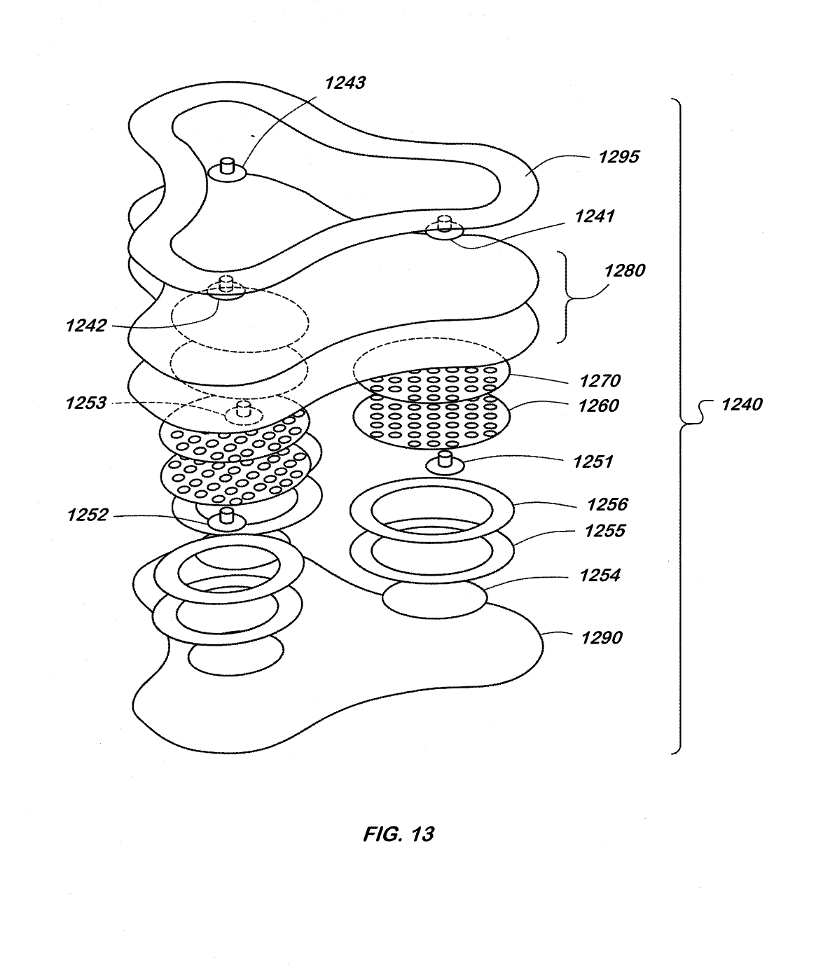

FIG. 13 is an exploded view of the adhesive patch 1240 portion of the biological sample collection system 1100 shown in FIG. 12. The adhesive patch 1240 includes upper studs 1241, 1242 and 1243, as described above. These studs are in electrical contact with the skin contact studs 1251, 1252, and 1253. On the skin side surface of the skin contact studs 1251, 1252, and 1253 is a conductive hydrogel layer 1254. Around each stud 1251, 1252, and 1253 are non-conductive hydrogel 1255 and pressure sensitive adhesive 1256 components. In this portion, any convenient physiologically acceptable adhesive may be employed. In some instances, adhesive that change their adhesive properties in response to an applied stimulus are employed. For example, adhesives that become less adhesive upon application of light, e.g., UV light, or a chemical, may be employed, so that the adhesive remains strong while it is desired for the receiver to remain associated with the body but is readily weakened to facilitate removal of the receiver from the body when desired. On the non-skin side of each skin contact stud is a layer of dry electrode material, such as Ag/AgCl. On the upper surface of this layer of dry electrode material is a porous layer, such as a carbon vinyl layer. Also shown are upper backing layers 1280. Though not shown, upper studs 1241 to 1243 are in electrical contact through the backing layers 1280 (for example urethane and polyethylene) with the dry electrode and skin contact studs which are positioned beneath each upper stud. As illustrated, the studs are off center with respect to their dry electrode layer in the direction of the outer edge of the patch in a manner sufficient to increase dipole size between any two given studs. In addition, where desired a conductivity gradient may be associated with each stud, e.g., by altering the pattern of the porous layer 1270 and/or modifying the composition of the dry electrode layer. Of interest in such aspects is where a conductivity gradient increases in conductivity in the direction of the outer edge of the patch. The biological sample collection module 1290 contacts the body of the subject to collect biological samples, which can later be analyzed at the processing facility as described above.

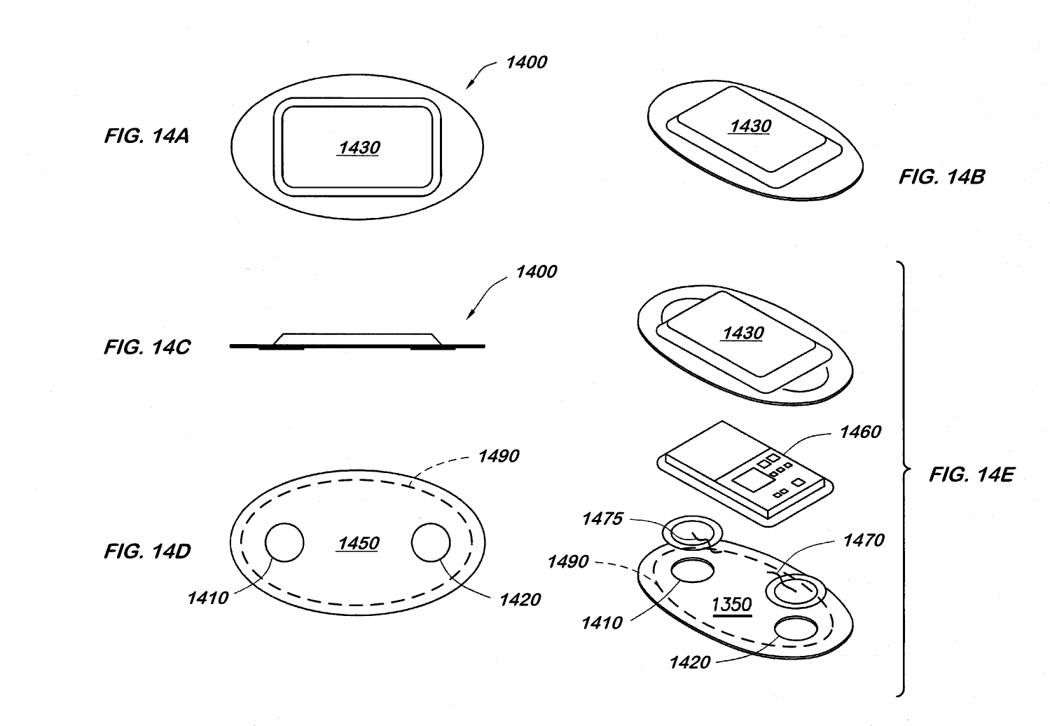

FIGS. 14A to 14E illustrate various views of an alternative external biological sample collection system 1400 configuration which includes two electrodes 1410 and 1420 in a flexible structure having an adhesive bandage configuration. The biological sample collection system 1400 includes an upper flexible outer support 1430 and a bottom flexible support 1450 which fit together as shown in FIG. 14E to enclose an integrated circuit/battery component 1460 and electrodes 1410 and 1420. As shown in FIG. 14D, the bottom surfaces of electrodes 1410 and 1420 are exposed. As shown in FIG. 14E, electrodes 1410 and 1420 include lead elements 1475 and 1470 which provide for electrical contact between the electrodes and the integrated circuit/battery component 1460. Any convenient adhesive component may be employed, such as those described above. The flexible support 1450 comprises a biological sample collection module 1490, which may comprise features, in any suitable configuration and combination, similar to any one of the features of the biological sample collection devices 100, 300, 400, 500, 600, 700, 800, 1600 described in connection with respective FIGS. 1, 3-8, and 16.

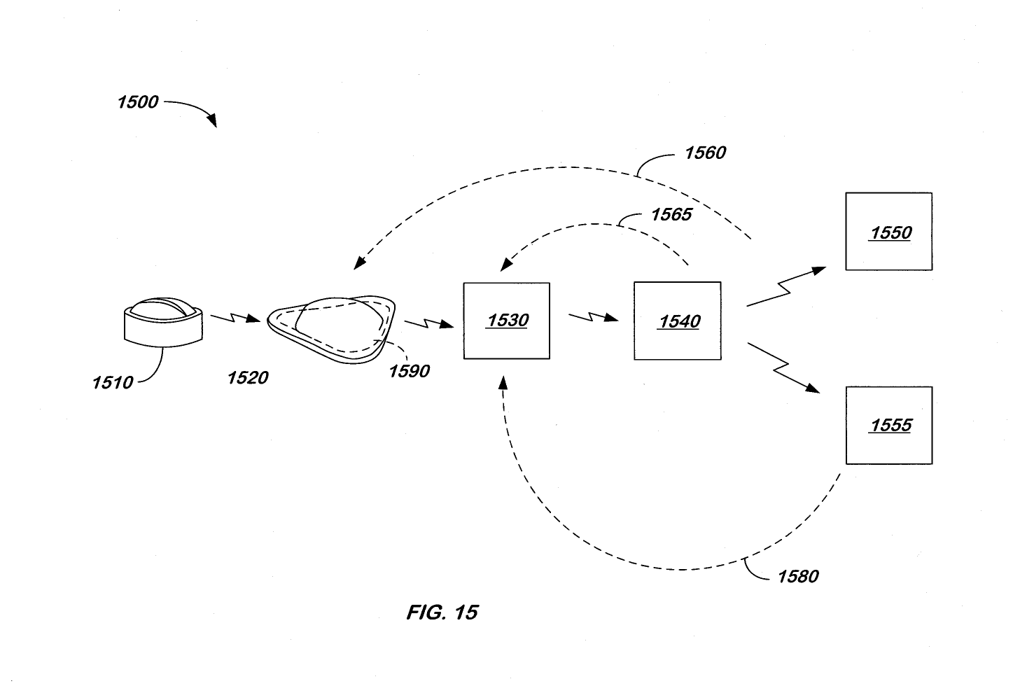

FIG. 15 illustrates one aspect of a communication system 1500 for a biological sample collection system. As shown in FIG. 15, the system 1500 includes a pharmaceutical composition 1510 that comprises an IEM. Also present in the system 1500 is a receiver 1520, such as the receiver 901, 1000 illustrated in FIGS. 9-10. The receiver 1520 comprises a biological sample collection portion 1590, which may comprise features, in any suitable configuration and combination, similar to any one of the features of the biological sample collection devices 100, 300, 400, 500, 600, 700, 800, 1600 described in connection with respective FIGS. 1, 3-8, and 16. The receiver 1520 is configured to detect a communication emitted by the identifier of the IEM 1510 whereas the biological sample collection portion 1590 is configured to collect a biological sample to be analyzed at a processing facility at a later time. In one aspect, the receiver 1520 also may include physiologic sensing capability, such as ECG and movement sensing capability. The receiver 1520 is configured to communicate data to an external communication device 1530 (such as a cell phone, smart phone, PDA, or other wireless communication enabled device) of the subject 102 (FIGS. 1, 2), which in turn communicates the data to a server 1540. The server 1540 may be configured as desired, e.g., to provide for subject directed permissions. For example, the server 1540 may be configured to allow a family caregiver 1550 to participate in a therapeutic regimen of the subject 102, e.g., via an interface (such as a web interface) that allows the family caregiver 1550 to monitor alerts and trends generated by the server 1540, and provide support back to the subject, as indicated by arrow 1560. The server 1540 also may be configured to provide responses directly to the subject, e.g., in the form of subject alerts, subject incentives, as indicated by arrow 1565 which are relayed to the subject via the communication device 1530. The server 1540 also may interact with a health care professional (e.g., RN, physician) 1555, which can use data processing algorithms to obtain measures of health and compliance of the subject, e.g., wellness index summaries, alerts, cross-patient benchmarks, and provide informed clinical communication and support back to the subject, as indicated by arrow 1580.

The body-associate biological sample collection devices and systems described herein (e.g., 100, 300, 400, 500, 600, 700, 800, 900, 1000, 1100, 1400, 1500) of interest include both external and implantable devices. In external aspects, the biological sample collection device is ex vivo, by which is meant that the device is present outside of the body during use. Where the biological sample collection devices are external, they may be configured in any convenient manner, where in certain aspects they are configured to be associated with a desirable skin location. As such, in certain aspects the external biological sample collection devices are configured to be contacted with a topical skin location of a subject. Configurations of interest for the biological sample collection devices described herein (e.g., 100, 300, 400, 500, 600, 700, 800, 900, 1000, 1100, 1400, 1500) include, but are not limited to: patches, wrist bands, jewelry (such as watches, earrings and bracelets), clothing, accessories, e.g., belts and shoes, eyeglasses. In some instances, the receivers are configured to adhere to a skin location, e.g., by use of suitable adhesive, such as described below. In some instances, the receivers are configured to touch a skin location but not adhere thereto, for example where the device is configured as a wrist band, an item of jewelry (such as a watch, an earring and a bracelet), an article of clothing, an accessory, such as a belt and a shoe, and a pair of eyeglasses. In yet other instances, the receivers may be configured to be maintained within some defined distance of a skin surface, such as within 1 cm, including within 0.5 cm.

In certain aspects, the biological sample collection devices and systems described herein (e.g., 100, 300, 400, 500, 600, 700, 800, 900, 1000, 1100, 1400, 1500) are implantable components. By implantable is meant that the biological sample collection device is designed, i.e., configured, for implantation into a subject, e.g., on a semi-permanent or permanent basis.

In these aspects, the biological sample collection devices and systems described herein (e.g., 100, 300, 400, 500, 600, 700, 800, 900, 1000, 1100, 1400) are in vivo during use. By implantable is meant that the biological sample collection device are configured to maintain functionality when present in a physiological environment, including a high salt, high humidity environment found inside of a body, for two or more days, such as about one week or longer, about four weeks or longer, about six months or longer, about one year or longer, e.g., about five years or longer. In certain aspects, the implantable biological sample collection devices are configured to maintain functionality when implanted at a physiological site for a period ranging from about one to about eighty years or longer, such as from about five to about seventy years or longer, and including for a period ranging from about ten to about fifty years or longer. For implantable aspects, the biological sample collection device may have any convenient shape, including but not limited to: capsule-shaped, disc-shaped. The biological sample collection device may be configured to be placed in a number of different locations, e.g., the abdomen, small of the back, shoulder (e.g., where implantable pulse generators are placed). In certain implantable aspects, the biological sample collection device is a standalone device, in that it is not physically connected to any other type of implantable device. In yet other aspects, the biological sample collection device may be physically coupled to a second implantable device, e.g., a device which serves as a platform for one or more physiological sensors, where the device may be a lead, such as a cardiovascular lead, where in certain of these aspects the cardiovascular lead includes one or more distinct physiological sensors, e.g., where the lead is a multi-sensor lead (MSL). Implantable devices of interest further include, but are not limited to: implantable pulse generators (e.g., ICDs), neurostimulator devices, implantable loop recorders.

The biological sample collection systems 900, 1000, 1100, 1400, 1500 may include a receiver element which serves to receive the conductive communication from the IEM. The receiver portion of the biological sample collection devices 900, 1000, 1100, 1400, 1500 may include a variety of different types of receiver elements, where the nature of the receiver element necessarily varies depending on the nature of the produced by the generation element. In certain aspects, the receiver element may include one or more electrodes for detecting communications from the generation element, such as two or more electrodes, three or more electrodes. In certain aspects, the receiver device will be provided with two or three electrodes that are dispersed at some distance from each other. This distance allows the electrodes to detect a differential voltage. The distance may vary, and in certain aspects ranges from 0.1 cm to 1.0 m, such as 0.1 to 5 cm, such as 0.5 to 2.5 cm, where the distance 1 cm in some instances.

The biological sample collection devices described herein may be able to collect environmental sample such as for air samples for mine workers or smog. So it is contemplated that any of the biological sample collection devices or systems 100, 300, 400, 500, 600, 700, 800, 900, 1000, 1100, 1400, 1500 disclosed herein could be configured to sample both the environment as well the subject 102.

In term of size, any of the biological sample collection devices or systems 100, 300, 400, 500, 600, 700, 800, 900, 1000, 1100, 1400, 1500 disclosed herein can be as small as a square centimeter and as large as 100 to 200 centimeters on a side. Accordingly, in various aspects, the size of the biological sample collection device may be range from about 1 cm to about 200 cm, for example.

Additional disclosure of receivers that may be employed in combination with the biological sample collection devices discussed herein is provided in U.S. patent application Ser. No. 12/673,326, titled “BODY-ASSOCIATED SIGNAL RECEIVER AND METHOD,” filed on Feb. 12, 2010, which is incorporated herein by reference in its entirety.

It is to be understood that this disclosure is not limited to particular aspects or aspects described, as such may vary. It is also to be understood that the terminology used herein is for the purpose of describing particular aspects or aspects only, and is not intended to be limiting, since the scope of the biological sample collection devices and systems is defined only by the appended claims.

Notwithstanding the claims, the invention is also defined by the following clauses:
1. A biological sample collection device, comprising:

a top cover plate;

a bottom portion attached to the top cover plate, the bottom portion comprising a remotely-analyzable biological sample collection portion to collect a biological sample from a body of a subject, wherein the biological sample is to be analyzed at a remote processing facility at a later time;

a fastening portion provided on the bottom portion to affix the remotely-analyzable biological sample collection device to the body of the subject;

2. The biological sample collection device of clause 1, comprising a real time clock coupled to the top cover plate.
3. The biological sample collection device of clause 2, comprising a memory coupled to the top cover plate and electrically coupled to the real time clock.
4. The biological sample collection device of clause 3, wherein the memory coupled to the real time clock is operative to time-stamp when the biological sample collection device is applied to the body of the subject
5. The biological sample collection device according to any of the preceding clauses wherein the remotely-analyzable biological collection portion comprises a sorbent material.
6. The biological sample collection device of clause 5, wherein the sorbent material comprises an adsorbent material or an absorbent material.
7. The biological sample collection device according to any of the preceding clauses wherein the remotely-analyzable biological collection portion comprises one or more of the following:

an adhesive material

at least one micro-needle.

a color indicator to indicate a presence or absence of a substance.

8. The biological sample collection according to any of the preceding clauses wherein the fastening portion comprises an adhesive material.
9. The biological sample collection device according to any of the preceding clauses further comprising:

at least two electrodes; and

an electronic module to extract fluids from the body of the subject via reverse electrophoresis and drive the biological sample from the body of the subject into the sample collection portion.

10. The biological sample collection device according to any of the preceding clauses further comprising a framework comprising a first and second dissimilar materials to generate an operational voltage when exposed to a conductive fluid when the biological collection device is applied to the body of the subject.
11. A biological sample collection system comprising a device according to any of the preceding clauses and further comprising:

a processing unit;

at least two electrodes coupled to the processing unit, the at least two electrodes configured to contact skin of a subject; and

a transbody conductive communication module coupled to the processing unit and the at least two electrodes, the transbody conductive communication module operative to detect and gather physiological information from the subject in the form of an electric current flow through the at least two electrodes at a first frequency, wherein the current flow at the first frequency is associated with a device associated with the subject.

12. The biological sample collection system of clause 11, further comprising:

a physiological sensing module coupled to the processing unit and the at least two electrodes, the physiological sensing module operative to sense physiological information from the subject in the form of electric current flow through the at least two electrodes at the second frequency, wherein the second frequency current flow is associated with the physiology of the subject.

13. The biological sample collection system of clause 11 or 12 further comprising:

a wireless communication module coupled to the processing unit operative to communicate information from the remotely-analyzable biological collection device to a communication device external to the subject.

14. The biological sample collection system according to any of the clauses 11-13 wherein the transbody conductive communication module is configured to receive communications from an ingestible event marker located inside the body of the subject.
15. The biological sample collection system according to any of the clauses 12-14 wherein the physiological sensing module is configured to receive communications from a device embedded in the body of the subject.
16. The biological sample collection system according to any of the clauses 13-15 wherein the wireless communication module is configured to communicate data obtained from the ingestible event marker, and/or wherein the wireless communication module is configured to receive data providing for subject directed permissions.
17. Use of a device and/or a system according to any of the preceding clauses for obtaining and/or transmitting physiological information from and/or to a subject

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges and are also encompassed within the present disclosure, subject to any specifically excluded limit in the stated range. Where the stated range includes one or both of the limits, ranges excluding either or both of those included limits also are included in the present disclosure.

Unless defined otherwise, all technical and scientific terms used herein have the same meaning as commonly understood by one of ordinary skill in the art to which the present disclosure belongs. Although any methods and materials similar or equivalent to those described herein can also be used in the practice or testing of the apparatuses, systems, and methods described in the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of priority. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as solely, only and the like in connection with the recitation of claim elements, or use of a negative limitation.

As will be apparent to those of skill in the art upon reading the present disclosure, each of the individual aspects and aspects described and illustrated herein has discrete components and features which may be readily separated from or combined with the features of any of the other several aspects without departing from the scope of the present disclosure. Any recited method can be carried out in the order of events recited or in any other order which is logically possible.

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the scope of the appended claims.

Accordingly, it will be appreciated that those skilled in the art will be able to devise various arrangements which, although not explicitly described or shown herein, embody the principles of the present disclosure and are included within its scope. Furthermore, all examples and conditional language recited herein are principally intended to aid the reader in understanding the principles described in the present disclosure and the concepts contributed to furthering the art, and are to be construed as being without limitation to such specifically recited examples and conditions. Moreover, all statements herein reciting principles, aspects, and aspects as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary aspects and aspects shown and described herein. Rather, the scope of present disclosure is embodied by the appended claims.

It is worthy to note that any reference to “one aspect” or “an aspect” means that a particular feature, structure, or characteristic described in connection with the aspect is included in at least one aspect. Thus, appearances of the phrases “in one aspect” or “in an aspect” in various places throughout the specification are not necessarily all referring to the same aspect. Furthermore, the particular features, structures or characteristics may be combined in any suitable manner in one or more aspects.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

While certain features of the aspects have been illustrated as described herein, many modifications, substitutions, changes and equivalents will now occur to those skilled in the art. It is therefore to be understood that the appended claims are intended to cover all such modifications and changes as fall within the scope of the present disclosure.

1. A biological sample collection device, comprising:
a top cover plate;
a bottom portion attached to the top cover plate, the bottom portion comprising a remotely-analyzable biological sample collection portion to collect a biological sample from a body of a subject, wherein the biological sample is to be analyzed at a remote processing facility at a later time;
a fastening portion provided on the bottom portion to affix the remotely-analyzable biological sample collection device to the body of the subject;

2. The biological sample collection device of claim 1, comprising a real time clock coupled to the top cover plate.

3. The biological sample collection device of claim 2, comprising a memory coupled to the top cover plate and electrically coupled to the real time clock.

4. The biological sample collection device of claim 3, wherein the memory coupled to the real time clock is operative to time-stamp when the biological sample collection device is applied to the body of the subject

5. The biological sample collection device of claim 1, wherein the remotely-analyzable biological collection portion comprises a sorbent material.

6. The biological sample collection device of claim 5, wherein the sorbent material comprises an adsorbent material.

7. The biological sample collection device of claim 5, wherein the sorbent material comprises an absorbent material.

8. The biological sample collection device of claim 1, wherein the remotely-analyzable biological collection portion comprises an adhesive material.

9. The biological sample collection device of claim 1, wherein the remotely-analyzable biological collection portion comprises at least one micro-needle.

10. The biological sample collection device of claim 1, wherein the remotely-analyzable biological collection portion comprises a color indicator to indicate a presence or absence of a substance.

11. The biological sample collection device of claim 1, wherein the fastening portion comprises an adhesive material.

12. The biological sample collection device of claim 1, comprising:
at least two electrodes; and
an electronic module to extract fluids from the body of the subject via reverse electrophoresis and drive the biological sample from the body of the subject into the sample collection portion.

13. The biological sample collection device of claim 1, comprising:
a framework comprising a first and second dissimilar materials to generate an operational voltage when exposed to a conductive fluid when the biological collection device is applied to the body of the subject.

14. A biological sample collection system, comprising:
a top cover plate;
a bottom portion attached to the top cover plate, the bottom portion comprising a remotely-analyzable biological sample collection portion to collect a biological sample from the subject to be analyzed at a remote processing facility at a later time;
a fastening portion provided on the bottom portion to affix the remotely-analyzable biological sample collection device to the body of the subject;
a processing unit;
at least two electrodes coupled to the processing unit, the at least two electrodes configured to contact skin of a subject; and
a transbody conductive communication module coupled to the processing unit and the at least two electrodes, the transbody conductive communication module operative to detect and gather physiological information from the subject in the form of an electric current flow through the at least two electrodes at a first frequency, wherein the current flow at the first frequency is associated with a device associated with the subject.

15. The biological sample collection system of claim 14, comprising:
a physiological sensing module coupled to the processing unit and the at least two electrodes, the physiological sensing module operative to sense physiological information from the subject in the form of electric current flow through the at least two electrodes at the second frequency, wherein the second frequency current flow is associated with the physiology of the subject.

16. The biological sample collection system of claim 14, comprising:
a wireless communication module coupled to the processing unit operative to communicate information from the remotely-analyzable biological collection device to a communication device external to the subject.

17. The biological sample collection system of claim 14, wherein the remotely-analyzable biological sample collection portion comprises a sorbent material.

18. The biological sample collection system of claim 14, wherein the remotely-analyzable biological sample collection portion comprises an adhesive material.

19. The biological sample collection system of claim 14, wherein the remotely-analyzable biological sample collection portion comprises at least one micro-needle.

20. The biological sample collection system of claim 14, wherein the remotely-analyzable biological sample collection portion comprises a color indicator to indicate a presence or absence of a substance.

21. A biological sample collection system, comprising:
a top cover plate;
a bottom portion attached to the top cover plate, the bottom portion comprising a remotely-analyzable biological sample collection portion to collect a biological sample from the subject to be analyzed at a remote processing facility at a later time;
a fastening portion provided on the bottom portion to affix the remotely-analyzable biological sample collection device to the body of the subject;
a processing unit;
at least two electrodes coupled to the processing unit, the at least two electrodes configured to contact skin of a subject;
a transbody conductive communication module coupled to the processing unit and the at least two electrodes, the transbody conductive communication module operative to detect and gather physiological information from the subject in the form of an electric current flow through the at least two electrodes at a first frequency, wherein the current flow at the first frequency is associated with a device associated with the subject;
a physiological sensing module coupled to the processing unit and the at least two electrodes, the physiological sensing module operative to sense physiological information from the subject in the form of electric current flow through the at least two electrodes at a second frequency, wherein the current flow at the second frequency is associated with the physiology of the subject; and
a wireless communication module coupled to the processing unit operative to communicate information from the biological collection device to a communication device external to the subject.

22. The biological sample collection system of claim 21, wherein the transbody conductive communication module is configured to receive communications from an ingestible event marker located inside the body of the subject.

23. The biological sample collection system of claim 21, wherein the physiological sensing module is configured to receive communications from a device embedded in the body of the subject.

24. The biological sample collection system of claim 22, wherein the wireless communication module is configured to communicate data obtained from the ingestible event marker.

25. The biological sample collection system of claim 24, wherein the wireless communication module is configured to receive data providing for subject directed permissions.
